(19) United States
(12) Patent Application Publication
(10) Pub. No.: US 2019/0224341 A1
(43) Pub. Date: Jul. 25, 2019

(54) RENAL CLEARABLE ORGANIC NANOCARRIERS

(71) Applicant: The General Hospital Corporation, Boston, MA (US)

(72) Inventors: Hak Soo Choi, Needham, MA (US); Homan Kang, Revere, MA (US); Georges El Fakhri, Brookline, MA (US)

(21) Appl. No.: 16/314,149

(22) PCT Filed: Jun. 29, 2017
(86) PCT No.: PCT/US2017/039896

§ 371 Date: Dec. 28, 2018

Related U.S. Application Data
(60) Provisional application No. 62/356,036, filed on Jun. 29, 2016.

(51) Int. Cl. A61K 49/00; A61K 47/69; A61K 47/64; A61K 31/506; A61P 35/00

(52) U.S. Cl. CPC A61K 49/0054; A61K 49/0056; A61K 49/0034; A61K 47/6951; A61K 47/64; B82Y 5/00; A61P 35/00; A61K 2123/00; A61K 47/6907; A61K 47/6911; A61K 31/506

(57) ABSTRACT

Disclosed herein are nanocarriers that include one or more cyclodextrin moieties conjugated to a polymer. The cyclodextrin moieties can complex therapeutic (e.g., anticancer) agents, and can be used to treat diseases such as cancer.

### CROSS REFERENCE TO RELATED APPLICATION

This application claims priority to U.S. Provisional Application No. 62/356,036 filed Jun. 29, 2016, the contents of which are hereby incorporated by reference.

### FEDERALLY SPONSORED RESEARCH OR DEVELOPMENT

This invention was made with Government support under Grant No. R01-EB-011523 awarded by the National Institutes of Health. The Government has certain rights in the invention.

### TECHNICAL FIELD

Provided herein are nanocarrier compounds useful for binding therapeutic agents (e.g., anticancer agents) to form complexes. Also provided are methods of using the complexes to treat cancer.

### BACKGROUND

Chemotherapy, targeted therapy, radiation therapy, and hormonal therapy are commonly used methods in the prevention, diagnosis, and treatment of cancer (see, e.g., A. Gadducci, S. Cosio, A. R. Genazzani, Crit. Rev. Oncol. Hematol. 2006, 58, 242-256). However, chemotherapy agents currently in use are cytotoxic and often cause serious adverse effects including immunosuppression, myelosuppression, mucositis, and alopecia due to nonspecific uptake by the immune system and normal (i.e., non-neoplastic) cells (R. V. Chari, Adv. Drug Delivery Rev. 1998, 31, 89-104). As such, research has been conducted to develop chemotherapeutic formulations that are capable of delivering drugs selectively to cancerous regions, thus circumventing damage to healthy organs (R. Miller, C. Jacobs, O. Kayser, Adv. Drug Delivery Rev. 2001, 47, 3-19).

### SUMMARY

Provided herein is a nanocarrier, comprising one or more cyclodextrin moieties conjugated to a polymer.

In some embodiments, the polymer defines a micelle, a liposome, a nanosphere, a dendrimer, or a hollow shell. In some embodiments, the polymer comprises ϵ-polylysine, L-polylysine, polylactic acid, and poly(lactic-co-glycolic acid), polyaspartic acid, polyglutamic acid, or polyglutamic acid-poly(ethylene glycol) copolymer.

In some embodiments, the cyclodextrin moiety is derived from a-cyclodextrin, β-cyclodextrin, γ-cyclodextrin, 2-hydroxypropyl-β-cyclodextrin, 2-hydroxypropyl-γ-cyclodextrin, methyl-β-cyclodextrin, a β-cyclodextrin thioether, or a cyanoethylated β-cyclodextrin. In some embodiments, at least one cyclodextrin moiety is conjugated to an amino group of the polymer. In some of these embodiments, the amino group is a terminal amino group.

In some embodiments, the nanocarrier further comprises a contrast agent, wherein the contrast agent is conjugated to the polymer. In some of these embodiments, the contrast agent comprises a near-infrared fluorophore. In some of these embodiments, the near-infrared fluorophore is selected from the group consisting of ZW800-1C, ZW800-1, ZW800-3C, ZW700-1, indocyanine green (ICG), Cy5, Cy5.5, Cy7, Cy7.5, IRDye800-CW (CW800), and ZWCC.

In some embodiments, the nanocarrier comprises one or more therapeutic agents that form a complex with the one or more cyclodextrin moieties. In some embodiments, the one or more therapeutic agents comprise an anticancer agent. In some embodiments, the one or more therapeutic agents are selected from the group consisting of afatinib, AG 879, alectinib (Alecensa), altiratinib, apatinib (Tykerb), ARQ-087, ARRY-112, ARRY-523, ARRY-651, AUY-922, AXD7451, AZ-23, AZ623, AZ64, AZD4547, AZD6918, AZD7451, BGJ398, binimetinib, BLU6864, BLU9931, brivatinib, cabozantinib, CEP-751 and CEP-701 (lestaurtinib), cetuximab (Erbitux), CH5183284, crizotinib (Xalkori),

CT327, dabrafenib (Tafinlar), danusertib, DCC-2036 (rebastinib), DCC-2157, dovitinib, DS-6051, encorafenib, erdafitinib, erlotinib, EWMD-2076, gefitinib (Iressa), GNF-4256, GNF-5837, Gö 6976, GTx-186, GW441756, imatinib (Gleevec), K252a, lapatinib, lenvatinib (Lenvima), Loxo-101, Loxo-195 (ARRY-656), lucitanib, LY2874455, MGCD516 (sitravatinib), motesanib, nilotinib (Tasigna), nintedanib, NVP-AST487, ONO-5390556, orantinib (TSU-68, panitumumab (Vectibix), pazopanib (Votrient), PD089828, PD166866, PD173074, pertuzumab (Perjeta), PF-477736, PHA-739358 (danusertib), PHA-848125AC (Milciclib), PLX7486, ponatinib (AP-24534), PZ-1, quercetin, regorafenib (Stivarga), RPI-1, ruxolitinib, RXDX101 (Entrectinib), RXDX105, semaxanib (SU5416), sorafenib, SPP86, SSR128129E, SU4984, SU5402, SU6668,

SUN11602, Sunitinib, TAS120, TG101209, TPX-0005, trastuzumab, TSR-011, vandetanib (Caprelsa), vatalanib, VSR-902A, and XL-184 (cabozantinib). For example, the one or more therapeutic agents comprise imatinib.

In some embodiments, the one or more therapeutic agents have a partition coefficient between water and n-octanol of at least about +1.0 (e.g., at least about +3.0 or at least about +6.0).

In some embodiments, the one or more therapeutic agents are conjugated with a fluorescent dye.

In some embodiments, the stoichiometric ratio of the cyclodextrin moiety to the therapeutic agent is 1:1. In some embodiments, the complex is stable at a pH of about 7.4. In some embodiments, the complex is unstable at a pH of lower than 7.0 (e.g., lower than about 6.0 or lower than about 5.0).

In some embodiments, at least about 60% of the therapeutic agent is released from the nanocarrier at a pH of about 5.0. In some embodiments, the complex dissociates following uptake of the nanocarrier into a tumor.

In some embodiments, the nanocarrier comprises one or more positively charged moieties. In some of these embodiments, the nanocarrier comprises from about 10 positively charged moieties to about 30 positively charged moieties (e.g., from about 20 positively charged moieties to about 28 positively charged moieties or about 24 positively charged moieties).

In some embodiments, the nanocarrier comprises one or more negatively charged moieties. In some of these embodiments, the nanocarrier comprises from about 10 negatively charged moieties to about 30 negatively charged moieties (e.g., from about 20 negatively charged moieties to about 28 negatively charged moieties or about 23 negatively charged moieties).

In some embodiments, the nanocarrier comprises one or more positively charged moieties and one or more negatively charged moieties. In some of these embodiments, the number of the one or more positively charged moieties is equal to the number of the one or more negatively charged moieties. In some embodiments, the nanocarrier comprises from about 8 to 14 positively charged moieties to about 8 to 14 negatively charged moieties. In some of these embodiments, the nanocarrier comprises about 12 positively charged moieties and about 12 negatively charged moieties.

In some embodiments, the nanocarrier has an overall positive charge. In some embodiments, the nanocarrier has an overall negative charge. In some embodiments, the nanocarrier has no charged moieties.

In some embodiments, the nanocarrier comprises an ammonium group. In some embodiments, the nanocarrier comprises a carboxylate group.

In some embodiments, the average molecular weight of the nanocarrier is from about 10,000 g/mol to about 22,000 g/mol (e.g., from about 13,000 g/mol to about 19,000 g/mol, about 16,000 g/mol, or about 17,000 g/mol).

In some embodiments, the nanocarrier comprises an average of from about 5 to about 8 cyclodextrin moieties. In some embodiments, the nanocarrier comprises an average of from about 8 to about 11 cyclodextrin moieties. In some embodiments, the nanocarrier comprises an average of from about 11 to about 14 cyclodextrin moieties. In some embodiments, the nanocarrier comprises an average of from about 6 to about 7 cyclodextrin moieties (e.g., an average of about 6.7 cyclodextrin moieties).

In some embodiments, the average hydrodynamic diameter of the nanocarrier is from about 1 nm to about about 5.5 nm (e.g., from about 4 to about 5 nm, or about 4.3, 4.4, 4.5, 4.6, 4.7, 4.8, or 4.9 nm).

In some embodiments, at least 30% of the nanocarrier is excreted in the urine after administration of the nanocarrier to a patient (e.g., at least 40%, at least 70%, at least 80%, or at least 90%).

In some embodiments, less than about 50% of the therapeutic agent is released in non-neoplastic cells after administration of the nanocarrier to a patient (e.g., less than about 30%, less than about 10%, less than about 5% of the therapeutic agent, less than about 2% of the therapeutic agent, less than about 1%).

In some embodiments, the patient is a human.

Also provided herein is a method of treating cancer in a patient, the method comprising administering a therapeutically effective amount of the nanocarrier of any of the preceding embodiments to the patient. In some embodiments, the cancer is selected from the group consisting of bladder cancer, lung cancer, brain cancer, melanoma, gastrointestinal cancer, breast cancer, non-Hodgkin lymphoma, cervical cancer, ovarian cancer, colorectal cancer, pancreatic cancer, esophageal cancer, prostate cancer, kidney cancer, skin cancer, leukemia, thyroid cancer, liver cancer, and uterine cancer. For example, the cancer is gastrointestinal cancer.

In some embodiments, the cancer is characterized by the presence of one or more solid tumors in the subject, and the uptake of the nanocarrier is higher to the one or more solid tumors than to any other organ or tissue type in the subject after administration. In some of these embodiments, the any other organ or tissue type is selected from the group consisting of the duodenum, the bladder, the heart, the intestine, the kidneys, the liver, the lungs, muscle tissue, the pancreas, and the spleen. In some of these embodiments, the any other organ or tissue type is selected from the group consisting of the duodenum, the heart, the intestine, the liver, the lungs, muscle tissue, the pancreas, and the spleen.

Also provided herein is a method of imaging a tissue in a patient, comprising administering the nanocarrier of any of the preceding embodiments to the patient. In some of these embodiments, the tissue comprises cancer cells. In some of these embodiments, the tissue comprises kidney tissue, bladder tissue, or both.

Definitions

The term “complex” as used herein refers to the binding of two compounds (e.g., a nanocarrier and a therapeutic agent) by means of intermolecular forces that, under certain conditions, lasts greater than 1 second (e.g., greater than 2 seconds, 4 seconds, 10 seconds, 60 seconds 1 minute, 2 minutes, 5 minutes, 20 minutes, 30 minutes, 1 hour, 2 hours, 4 hours, 1 day, 3 days, 1 week, 2 weeks, 1 month, 2 months, 6 months, 1 year, 2 years, 5 years, or 10 years). For example, a complex formed between the cyclodextrin moiety of a nanocarrier and a therapeutic agent may be bound, in part, by hydrogen bonds between the hydroxyl groups of the cyclodextrin moiety and hydrogen bond accepting groups and/or atoms in the therapeutic agent.

The term “compound” as used herein is meant to include all stereoisomers, geometric isomers, tautomers, and isotopes of the structures depicted. Compounds herein identified by name or structure as one particular tautomeric form are intended to include other tautomeric forms unless otherwise specified. Compounds include, but are not limited to, the nanocarriers and therapeutic agents described herein.

Compounds provided herein also include tautomeric forms. Tautomeric forms result from the interchange of a single bond with an adjacent double bond together with the concomitant migration of a proton. Tautomeric forms include prototropic tautomers which are isomeric protonation states having the same empirical formula and total charge. Example prototropic tautomers include ketone-enol pairs, amide-imidic acid pairs, lactam-lactim pairs, enamine-imine pairs, and annular forms where a proton can occupy two or more positions of a heterocyclic system, for example, 1H- and 3H-imidazole, 1H-, 2H- and 4H-1,2,4-triazole, 1H- and 2H-isoindole, and 1H- and 2H-pyrazole. Tautomeric forms can be in equilibrium or sterically locked into one form by appropriate substitution.

As used herein, the term “defines,” refers to the three-dimensional form a compound (e.g., a polymer) assumes. For example, a polymer can assume a micelle, a liposome, a nanosphere, a dendrimer, or a hollow shell.

As used herein, the term “derived from” refers to when a moiety is structurally identical in most respects to the compound it is derived from. In some embodiments, the compound that the moiety is derived from was used as a reagent or intermediate in the synthesis of the compound that is substituted with the moiety. In some embodiments, the moiety only differs structurally from the compound it is derived from at the portion of the moiety that links to the remainder of the molecule that the moiety substitutes.

As used herein, the term “dissociates” refers to the process wherein the intermolecular forces between two compounds (e.g., a nanocarrier and a therapeutic agent) that form a complex break. For example, the dissociation of an inclusion complex between the cyclodextrin moiety of a nanocarrier and a therapeutic agent may include the breaking of hydrogen bonds between the cyclodextrin moieties and the nanocarrier.

As used herein, the term “non-neoplastic cell,” refers to a cell that is not a cancer cell and that occurs normally in the tissues, organs, and bodily fluids of an organism.

As used herein, the term “partition coefficient” refers to the ratio of the concentrations of a solute (e.g., a therapeutic agent) in two immiscible or slightly miscible phases (e.g., two liquid phases, e.g., water and n-octanol), when the solute is in equilibrium across the interface between the two phases.

As used herein, the term “patient,” refers to any animal, including mammals (e.g., domesticated mammals). Example patients include, but are not limited to, mice, rats, rabbits, dogs, cats, swine, cattle, sheep, horses, primates, and humans.

As used herein, the term “stable,” refers to a complex that does not readily dissociate when in a particular in vivo environment. For example, a complex comprising a nanocarrier provided herein and a therapeutic agent is stable at neutral pH and as such does not dissociate to a significant degree when in the bloodstream prior to uptake into a cancer cell (e.g., less than 5% or less than 1% of the complex dissociates). As used herein, the term “unstable” refers to a complex that dissociates to a significant degree when in a particular in vivo environment. For example, a complex comprising a nanocarrier provided herein and a therapeutic agent is unstable at acidic pH (e.g., about pH 5.0) and as such dissociates to a significant degree (e.g., more than 30% or more than 60% of the complex dissociates) after uptake into a cancer cell (i.e., an acidic environment).

As used herein, the term “zwitterion” refers to a group comprising one or more positively charged groups (e.g., ammonium) and one or more negatively charged groups (e.g., carboxylate).

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

### DESCRIPTION OF DRAWINGS

FIG. 1 is a graphical representation of an exemplary nanocarrier.

FIG. 2 depicts molecular structures of portions of ϵ-polylysine that include charged moieties.

FIGS. 3A and 3B are ¹H NMR spectra of ϵ-polylysine (FIG. 3A) and a β-cyclodextrin moiety (FIG. 3B).

FIGS. 4A-4C depict a size-exclusion chromatogram of nanocarrier 6 (FIG. 4A), superimposed plots of absorbance and fluorescence spectra of nanocarrier 6 (FIG. 4B); and superimposed plots of fluorescence and absorbance of nanocarrier 6 in fetal bovine serum over time (FIG. 4C).

FIGS. 5A and 5B depict plots of absorbance vs. wavelength of each product of a series of reactions of succinic anhydride (SA) with nanocarrier 6 (FIG. 5A); and a plot of percentage conversion of the amino groups in nanocarrier 6 vs. the molar ratio of SA to each lysine unit of nanocarrier 6 used in the reactions (FIG. 5B).

FIGS. 6A-6C depict a calibration curve of absorbance as a function of amount of ϵ-polylysine (FIG. 6A); photographic images of nanocarriers 6, 7, 8, and 9 after ninhydrin treatment (FIG. 6B); and a plot of absorbance vs. wavelength for each nanocarrier (FIG. 6C).

FIGS. 7A-7D depict histological photographic images and near-infrared (NIR) fluorescence signals of nanocarriers 6, 7, 8, and 9 at four hours after injection into xenograft mouse models in vivo (FIG. 7A) and after resection (FIG. 7B); the signal-to-background ratio (SBR) of organs vs. muscle for each nanocarrier at four hours after injection into xenograft mouse models (FIG. 7C); and a diagram of the hypothesized distribution and elimination pathway of the nanocarriers (FIG. 7D).

FIGS. 8A-8D depict blood concentration curves of nanocarriers 6, 7, 8, and 9 after intravenous injection (FIG. 8A); a bar graph of the half-life of each nanocarrier after injection (FIG. 8B); a bar graph of the percentage of each nanocarrier renally excreted (FIG. 8C); and a bar graph of plasma clearance and volume of distribution of each nanocarrier (FIG. 8D).

FIGS. 9A-9C depict molecular structures of nanocarrier 7 and imatinib conjugated to Cy3 fluorescent dye (FIG. 9A); a plot of absorbance vs. wavelength of Cy3-imatinib, ZW800-CDPL, and a complex of nanocarrier 7 and Cy3-imatinib (FIG. 9B); and the percentage of the Cy3-imatinib released from its complex with nanocarrier 7 as a function of time at pH 5.0 and 7.4 (FIG. 9C).

FIGS. 10A-10D depict a plot of tumor-to-background ratio over time of the 7-Cy3-imatinib complex after intravenous injection into gastrointestinal tumor (GIST)-bearing xenograft mice with MR fluorescence images at 3 different time points (FIG. 10A); color and MR fluorescence images of organs in vivo 24 h after injection (FIG. 10B); photographic and MR fluorescence images of organs ex vivo 24 h after injection and a bar graph of TBR of several organs (FIG. 10C); and histopathological images of resected tumor that includes a stain, the Cy3-imatinib conjugate, nanocarrier 7, and an overlay of the first two images (FIG. 10D).

FIGS. 11A-11C depict photographic images of gastrointestinal tumor (GIST)-bearing xenograft mice after intravenous injection of the 7-Cy3-imatinib complex at various time points after injection (FIG. 11A); photographic and MR fluorescence images of resected organs from xenograft mice 24 h after the intravenous injection (FIG. 11B); and photographic and MR fluorescence images of resected organs from transgenic mice 24 h after the intravenous injection (FIG. 11C).

FIGS. 12A and 12B depict photographic and MR fluorescence images of organs of CD-1 mice 4 h after injection of ZW800-1C contrast agent (FIG. 12A), and a bar graph of the signal-to-background ratio of each organ against muscle (FIG. 12B).

FIGS. 13A and 13B depict photographic and MR fluorescence images of organs of CD-1 mice 4 h after injection of ZW800 contrast agent conjugated to imatinib (FIG. 13A), and a bar graph of the signal-to-background ratio of each organ against muscle (FIG. 13B).

### DETAILED DESCRIPTION

Most conventional chemotherapeutic agents currently in use passively target cancer cells, thus causing side effects due to their nonspecific uptake into normal (i.e., non-neoplastic) cells. Accordingly, there is a need for agents that actively and selectively target cancer cells with minimal interactions with normal tissue and organs. Herein are disclosed nanocarriers that bind a therapeutic agent (e.g., anticancer agent) and, after administration to a subject, selectively deliver the therapeutic agent to cancer cells. The pharmacokinetics of the therapeutic agent can be controlled by binding the therapeutic agent to the nanocarriers, resulting in one or more of: i) protection of the therapeutic agent from unwanted degradation, ii) prevention of nonspecific interactions between the therapeutic agent and non-neoplastic cells, iii) enhancement of therapeutic agent absorption into the target tissue, and (iv) rapid excretion (e.g., renal clearance) from the body and/or an efficient degradation into nontoxic products. In some embodiments, cellular internalization and metabolism of the nanocarrier and its payload are limited, thus effectively minimizing exposure of the immune system and bodily tissues to the nanocarrier and its payload.

### Nanocarriers

The nanocarriers disclosed herein include one or more cyclodextrin moieties conjugated to a polymer. A polymer as described herein is biocompatible (i.e., non-toxic). In some embodiments, the polymer defines a micelle, a liposome, a nanosphere, a dendrimer, or a hollow shell. In some embodiments, the polymer includes a polypeptide, a polyester, and/or a derivative thereof. In certain embodiments, the polymer includes ϵ-polylysine, L-polylysine, polylactic acid, poly(lactic-co-glycolic acid), polyaspartic acid, polyglutamic acid, polyglutamic acid-poly(ethylene glycol) copolymer, any derivative thereof, or any combination thereof. In certain embodiments, the polymer is ϵ-polylysine, L-polylysine, polylactic acid, poly(lactic-co-glycolic acid), polyaspartic acid, polyglutamic acid, polyglutamic acid-poly(ethylene glycol) copolymer, or any derivative thereof. For example, the polymer is ϵ-polylysine.

In some embodiments, the cyclodextrin moiety is derived from a-cyclodextrin, β-cyclodextrin, γ-cyclodextrin, 2-hydroxypropyl-β-cyclodextrin, 2-hydroxypropyl-γ-cyclodextrin, methyl-β-cyclodextrin, a β-cyclodextrin thioether, or a cyanoethylated β-cyclodextrin. For example, the cyclodextrin moiety is derived from β-cyclodextrin. In some embodiments, at least one (e.g., at least 2, 3, 4, 5, 6, 7, 8, 9, 10, 11, 12, 15, 20, or 25) cyclodextrin moiety is conjugated to an amino group (e.g., an α-amino group or a terminal amino group) of the polymer. In certain of these embodiments, at least one cyclodextrin moiety is conjugated to an amino group of the polymer from the 6′-position of a hexose unit of the cyclodextrin moiety.

In some embodiments, the nanocarrier includes a contrast agent conjugated to the polymer. In some embodiments, the contrast agent is conjugated to an amino group (e.g., an a-amino group or a terminal amino group) of the polymer. In certain of these embodiments, the contrast agent is conjugated to an amino group of the polymer by an amido linkage.

In some embodiments, the contrast agent includes a fluorophore (e.g., a near-infrared fluorophore (NIRF)). In some embodiments, the near-infrared fluorophore is selected from the group consisting of ZW800 (e.g., ZW800-1C, ZW800-1, or ZW800-3C), ZW700-1, indocyanine green (ICG), CyS, Cy5.5, Cy7, Cy7.5, IRDye800-CW (CW800), and ZWCC. In certain of these embodiments, the near-infrared fluorophore is ZW800 (e.g., ZW800-1C, ZW800-1, or ZW800-3C).

In some embodiments, the nanocarrier includes one or more therapeutic agents that form a complex (i.e., a host-guest or inclusion complex) with the one or more cyclodextrin moieties. In certain embodiments, the one or more therapeutic agents include an anticancer agent. In certain embodiments, the one or more therapeutic agents is an anticancer agent. Exemplary therapeutic agents include afatinib, AG 879, alectinib (Alecensa), altiratinib, apatinib (Tykerb), ARQ-087, ARRY-112, ARRY-523, ARRY-651, AUY-922, AXD7451, AZ-23, AZ623, AZ64, AZD4547, AZD6918, AZD7451, BGJ398, binimetinib, BLU6864, BLU9931, brivatinib, cabozantinib, CEP-751 and CEP-701 (lestaurtinib), cetuximab (Erbitux), CH5183284, crizotinib (Xalkori), CT327, dabrafenib (Tafinlar), danusertib, DCC-2036 (rebastinib), DCC-2157, dovitinib, DS-6051, encorafenib, erdafitinib, erlotinib, EWMD-2076, gefitinib (Iressa), GNF-4256, GNF-5837, Gö 6976, GTx-186, GW441756, imatinib (Gleevec), K252a, lapatinib, lenvatinib (Lenvima), Loxo-101, Loxo-195 (ARRY-656), lucitanib, LY2874455, MGCD516 (sitravatinib), motesanib, nilotinib (Tasigna), nintedanib, NVP-AST487, ONO-5390556, orantinib (TSU-68, panitumumab (Vectibix), pazopanib (Votrient), PD089828, PD166866, PD173074, pertuzumab (Perjeta), PF-477736, PHA-739358 (danusertib), PHA-848125AC (Milciclib), PLX7486, ponatinib (AP-24534), PZ-1, quercetin, regorafenib (Stivarga), RPI-1, ruxolitinib, RXDX101 (Entrectinib), RXDX105, semaxanib (SU5416), sorafenib, SPP86, SSR128129E, SU4984, SU5402, SU6668, SUN11602, Sunitinib, TAS120, TG101209, TPX-0005, trastuzumab, TSR-011, vandetanib (Caprelsa), vatalanib, VSR-902A, or XL-184 (cabozantinib). In some embodiments, the therapeutic agent is imatinib.

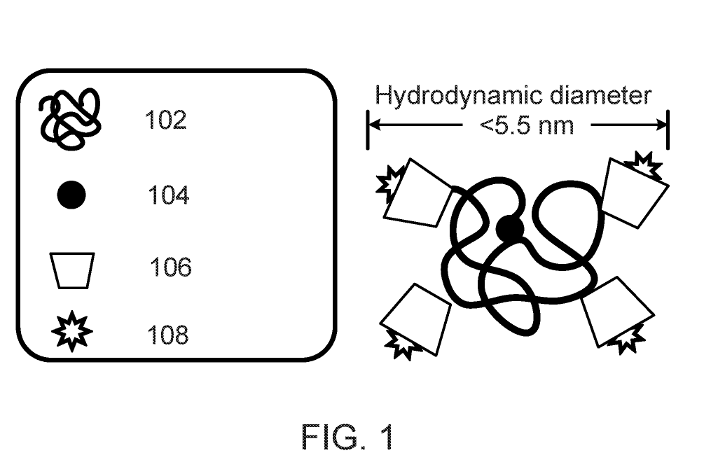

FIG. 1 is a representation of a nanocarrier that includes a polymer (102), a contrast agent conjugated to the polymer (104), cyclodextrin moieties conjugated to the polymer (106), and a molecule of therapeutic agent complexed to each cyclodextrin moiety (108).

In some embodiments, the therapeutic agent is hydrophobic. Without being bound by any theory, it is believed that the therapeutic agents should be hydrophobic in order to remain bound to the nanocarrier in vivo at or near neutral pH (i.e., about 7.0). In certain embodiments, a measure of the hydrophobicity of the therapeutic agent is its partition coefficient. In some embodiments, the one or more therapeutic agents have a partition coefficient between water and n-octanol of at least about +1.0 (e.g., at least about +1.5, at least about +2.0, at least about +2.5, at least about +3.0, at least about +3.5, at least about +4.0, at least about +4.5, at least about +5.0, at least about +5.5, at least about +6.0, at least about +6.5, at least about +7.0, at least about +7.5, at least about +8.0, at least about +8.5, at least about +9.0, at least about +9.5, at least about +10.0, at least about +10.5, at least about +11.0, at least about +11.5, or at least about +12.0). In some embodiments, the one or more therapeutic agents have a partition coefficient between water and n-octanol of about +1.0, +1.5, +2.0, +2.5, +3.0, +3.5, +4.0, +4.5, +5.0, +5.5, +6.0, +6.5, +7.0, +7.5, +8.0, +8.5, +9.0, +9.5, +10.0, +10.5, +11.0, +11.5, or +12.0).

In some embodiments, the one or more therapeutic agents are conjugated with a fluorescent dye. In certain instances, conjugating a fluorescent dye to the therapeutic agent enables tracking (e.g., imaging) of the therapeutic agent in vivo. In some embodiments, the fluorescent dye includes a xanthene derivative (e.g., fluorescein, rhodamine, Oregon green, eosin, or Texas red), cyanine derivative (e.g., cyanine, indocarbocyanine, oxacarbocyanine, thiacarbocyanine, or merocyanine), squaraine derivative or ring-substituted squaraine (e.g., seta, setau, and square dyes), naphthalene derivative (e.g., dansyl or prodan derivatives), coumarin derivative, oxadiazole derivative (e.g., pyridyloxazole, nitrobenzoxadiazole, or benzoxadiazole), anthracene derivative (e.g., anthraquinones, including DRAQS, DRAQ7, or CyTRAK orange), pyrene derivative (e.g., cascade blue), oxazine derivative (e.g., nile red, nile blue, cresyl violet, or oxazine 170), acridine derivative (e.g., proflavin, acridine orange, or acridine yellow), arylmethine derivative (e.g., auramine, crystal violet, or malachite green), tetrapyrrole derivative (e.g., porphin, phthalocyanine, or bilirubin), ZW800 (e.g., ZW800-1C, ZW800-1, or ZW800-3C), ZW700-1, indocyanine green (ICG), CyS, Cy5.5, Cy7, Cy7.5, IRDye800-CW (CW800), or ZWCC. In some embodiments, the fluorescent dye is a xanthene derivative (e.g., fluorescein, rhodamine, Oregon green, eosin, or Texas red), cyanine derivative (e.g., cyanine, indocarbocyanine, oxacarbocyanine, thiacarbocyanine, or merocyanine), squaraine derivative or ring-substituted squaraine (e.g., seta, setau, and square dyes), naphthalene derivative (e.g., dansyl or prodan derivatives), coumarin derivative, oxadiazole derivative (e.g., pyridyloxazole, nitrobenzoxadiazole, or benzoxadiazole), anthracene derivative (e.g., anthraquinones, including DRAQ5, DRAQ7, or CyTRAK orange), pyrene derivative (e.g., cascade blue), oxazine derivative (e.g., nile red, nile blue, cresyl violet, or oxazine 170), acridine derivative (e.g., proflavin, acridine orange, or acridine yellow), arylmethine derivative (e.g., auramine, crystal violet, or malachite green), tetrapyrrole derivative (e.g., porphin, phthalocyanine, or bilirubin), ZW800 (e.g., ZW800-1C, ZW800-1, or ZW800-3C), ZW700-1, indocyanine green (ICG), CyS, Cy5.5, Cy7, Cy7.5, IRDye800-CW (CW800), or ZWCC.

In some embodiments, the stoichiometric ratio of the cyclodextrin moiety to the therapeutic agent is 10:1, 9:1, 8:1, 7:1, 6:1, 5:1, 4:1, 3:1, 2:1, or 1:1. In some embodiments, the stoichiometric ratio of the cyclodextrin moiety to the therapeutic agent is 1:1. In some embodiments, the complex formed between the cyclodextrin moiety and the therapeutic agent is stable at a pH of from about 7.0 to about 8.0 (e.g., at a pH of about 7.4 or 7.4). For example, the complex is stable at physiological pH (e.g., in the bloodstream). In some embodiments, the complex formed between the cyclodextrin moiety and the therapeutic agent is unstable at a pH of lower than 7.0 (e.g., lower than about 6.0 or 5.0). In some embodiments, the complex formed between the cyclodextrin moiety and the therapeutic agent is unstable at a pH of about 5.0 or 5.0. For example, the complex is unstable after uptake into cancer cells. In some embodiments, the complex dissociates following uptake of the nanocarrier into a tumor. In some embodiments, at least about 50% (e.g., at least about 60%, at least about 65%, at least about 70%, at least about 75%, at least about 80%, at least about 85%, at least about 90%, at least about 95%, at least about 98%, at least about 99%) of the therapeutic agent is released from the nanocarrier at a pH of about 5.0 and/or after uptake into cancer cells.

In some embodiments, the nanocarrier includes balanced nonsticky charges (i.e., zwitterionic or non-charged polar surface) at or near the surface of the nanocarrier (i.e., directly exposed to the physiological environment, e.g., bodily fluids, e.g., blood). Without being bound by any theory, it is believed that including balanced nonsticky charges can enhance nanocarrier selectivity by reducing nonspecific tissue uptake. In some embodiments, the nanocarrier includes one or more positively charged moieties. In some embodiments, the nanocarrier includes from about 10 positively charged moieties to about 30 positively charged moieties (e.g., about 10, 11, 12, 13, 14, 15, 16, 17, 18, 19, 20, 21, 22, 23, 24, 25, 26, 27, 28, 29, or 30 positively charged moieties). For example, the nanocarrier includes about 12 or 24 positively charged moieties. In some embodiments, the positively charged moieties include an ammonium group.

In some embodiments, the nanocarrier includes one or more negatively charged moieties. In some embodiments, the nanocarrier includes from about 10 negatively charged moieties to about 30 negatively charged moieties. In some embodiments, the nanocarrier includes from about 20 negatively charged moieties to about 28 negatively charged moieties. For example, the nanocarrier includes about 10, 11, 12, 13, 14, 15, 16, 17, 18, 19, 20, 21, 22, 23, 24, 25, 26, 27, 28, 29, or 30 negatively charged moieties. For example, the nanocarrier includes about 23 negatively charged moieties. In some embodiments, the negatively charged moieties include a carboxylate group.

In some embodiments, the nanocarrier includes one or more positively charged moieties and one or more negatively charged moieties (i.e., the nanocarrier is zwitterionic). In some embodiments, the number of the one or more positively charged moieties is equal to the number of the one or more negatively charged moieties. In some embodiments, the nanocarrier includes from about 8 to about 14 (e.g., 8, 9, 10, 11, 12, 13, or 14) positively charged moieties and from about 8 to about 14 (e.g., 8, 9, 10, 11, 12, 13, or 14) negatively charged moieties. In some embodiments, the nanocarrier includes about 12 positively charged moieties and about 12 negatively charged moieties. In some embodiments, the nanocarrier has an overall positive charge. In some embodiments, the nanocarrier has an overall negative charge.

In some embodiments, the nanocarrier has no charged moieties.

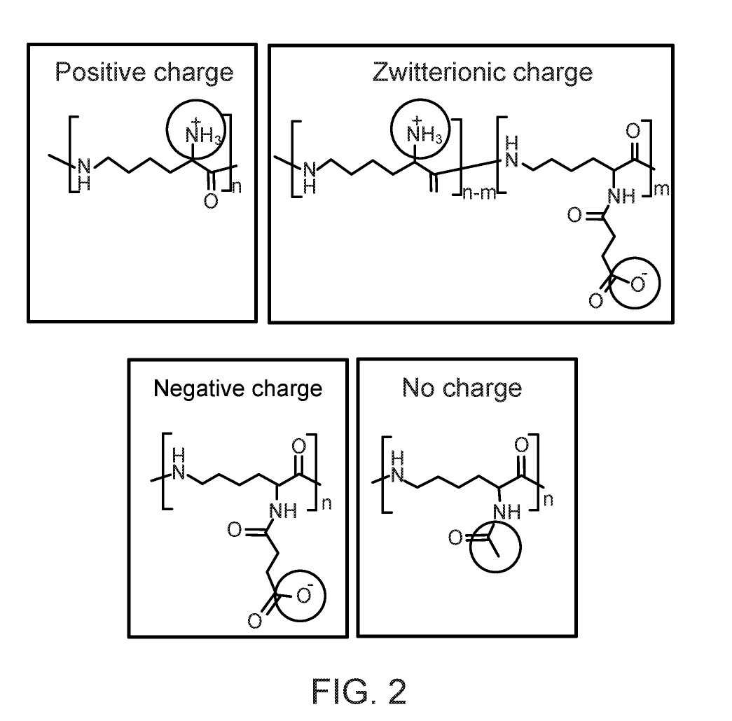

FIG. 2 depicts representative monomeric units of the polymer (ϵ-polylysine) that include a positively charged moiety (ammonium), both a positively charged moiety (ammonium) and a negatively charged moiety (carboxylate), a negatively charged moiety (carboxylate), and no charged moieties.

In some embodiments, the average molecular weight of the nanocarrier is from about 10,000 g/mol to about 22,000 g/mol (e.g., from about 10,000 g/mol to about 13,000 g/mol, from about 13,000 g/mol to about 15,000 g/mol, from about 15,000 g/mol to about 17,000 g/mol, from about 17,000 g/mol to about 19,000 g/mol, from about 19,000 g/mol to about 22,000 g/mol). For example, the average molecular weight of the nanocarrier is about 10,000 g/mol, about 11,000 g/mol, about 12,000 g/mol, about 13,000 g/mol, about 14,000 g/mol, about 15,000 g/mol, about 16,000 g/mol, about 17,000 g/mol, about 18,000 g/mol, about 19,000 g/mol, about 20,000 g/mol, about 21,000 g/mol, or about 22,000 g/mol. For example, the average molecular weight of the nanocarrier is about 16,000 g/mol or about 17,000 g/mol.

In some embodiments, the nanocarrier includes an average of from about 1 to about 30 cyclodextrin moieties (e.g., from about 5 to about 25, from about 5 to about 20, from about 5 to about 15, from about 5 to about 10, from about 5 to about 8, from about 6 to about 7, from about 8 to about 11, from about 11 to about 14, from about 14 to about 17, from about 17 to about 21, from about 21 to about 24, from about 24 to about 27, or from about 27 to about 30 cyclodextrin moieties). For example, the nanocarrier includes an average of about 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 cyclodextrin moieties. For example, the nanocarrier includes an average of about 6.7 cyclodextrin moieties.

In some embodiments, the hydrodynamic diameter of the nanocarrier is less than or about equal to 5.5 nm. Without being bound by any theory, it is believed that a hydrodynamic diameter of less than the threshold of glomerular filtration (i.e., about 5.5 nm) to enable renal clearance of the nanocarrier (e.g, following release of the therapeutic agent). In some embodiments, the average hydrodynamic diameter of the nanocarrier is from about 1 nm to about 5.5 nm (e.g., about 1 to 2, about 1 to 3, about 1 to 4, about 1 to 5, about 2 to 5, about 3 to 5, about 4 to 5, about 2 to 4, about 2 to 3, or about 3 to 4 nm). For example, the average hydrodynamic diameter of the nanocarrier is from about 4 to about 5 nm (e.g., about 4.3, 4.4, 4.5, 4.6, 4.7, 4.8, or 4.9 nm). In some embodiments, at least 30% (e.g, at least 40%, at least 50%, at least 60%, at least 70%, at least 80%, at least 90%, at least 95%, at least 98%, or at least 99%) of the nanocarrier (e.g., the nanocarrier after dissociation of therapeutic agent; or collectively the nanocarrier comprising therapeutic agent and the nanocarrier after dissociation of therapeutic agent) is excreted in the urine after administration of the nanocarrier to a patient (e.g., following release of the therapeutic agent). In some of these embodiments, the patient is a mammal (e.g., a human or a domesticated mammal).

In some embodiments, less than about 70% (e.g., less than about 60%, 50%, 40%, 30%, 20%, 10%, 7%, 5%, 2%, 1%) of the nanocarrier undergoes nonspecific uptake by non-neoplastic cells after administration of the nanocarrier to a patient (e.g., prior to release of the therapeutic agent and/or prior to excretion). In some of these embodiments, the patient is a mammal (e.g., a human or a domesticated mammal).

In some embodiments, less than about 70% (e.g., less than about 60%, 50%, 40%, 30%, 20%, 10%, 7%, 5%, 2%, 1%) of the therapeutic agent is released into non-neoplastic cells after administration of the nanocarrier to a patient. In some of these embodiments, the patient is a mammal (e.g., a human or a domesticated mammal).

### Methods of Use

The present application further provides methods of treating a disease or disorder in a patient (e.g., cancer), including administering a therapeutically effective amount of the nanocarrier provided herein to the patient. For example, a therapeutically effective amount of the nanocarrier can be determined based upon the amount of therapeutic agent to be administered to the patient by the nanocarrier. In some embodiments, the cancer is selected from the group consisting of bladder cancer, lung cancer, brain cancer, melanoma, gastrointestinal cancer, breast cancer, non-Hodgkin lymphoma, cervical cancer, ovarian cancer, colorectal cancer, pancreatic cancer, esophageal cancer, prostate cancer, kidney cancer, skin cancer, leukemia, thyroid cancer, liver cancer, and uterine cancer. For example, the cancer is gastrointestinal cancer.

In some embodiments, the cancer is characterized by the presence of one or more solid tumors in the subject. In certain of these embodiments, the uptake of the nanocarrier (e.g., the nanocarrier prior to release of the therapeutic agent) is higher to the one or more tumors than to any other organ or tissue type in the subject after administration of the nanocarrier (e.g., the nanocarrier prior to release of the therapeutic agent). In some embodiments, the any other organ or tissue type includes the duodenum, the bladder, the heart, the intestine, the kidneys, the liver, the lungs, muscle tissue, the pancreas, or the spleen. In certain of these embodiments, the any other organ or tissue type is selected from the group consisting of the duodenum, the heart, the intestine, the liver, the lungs, muscle tissue, the pancreas, and the spleen.

The present application further provides methods of imaging a tissue in a subject, including administering the nanocarrier provided herein to the patient. In some embodiments, the tissue includes cancer cells. In some embodiments, the tissue includes kidney tissue, bladder tissue, or both.

In some embodiments, the patient is a mammal (e.g., a human or a domesticated mammal).

### Pharmaceutical Compositions and Formulations

When employed as pharmaceuticals, the nanocarriers provided herein can be administered via various routes (e.g., intravenous, intranasal, intradermal, or oral administration) in the form of pharmaceutical compositions. These compositions can be prepared as described herein or elsewhere, and can be administered by a variety of routes, depending upon whether local or systemic treatment is desired and upon the area to be 1 0 treated. In some embodiments, the administration is parenteral. Parenteral administration includes, for example, intravenous, intraarterial, subcutaneous, intraperitoneal intramuscular or injection or infusion; or intracranial administration, (e.g., intrathecal or intraventricular, administration). Parenteral administration can be in the form of a single bolus dose, or may be, for example, by a continuous perfusion pump. In some embodiments, the compounds, salts, and pharmaceutical compositions provided herein are suitable for parenteral administration. In some embodiments, the nanocarriers provided herein are suitable for intravenous administration. Conventional pharmaceutical carriers, aqueous, powder or oily bases, thickeners and the like may be necessary or desirable.

Also provided are pharmaceutical compositions which contain, as the active ingredient, a nanocarrier provided herein (e.g., a nanocarrier comprising a therapeutic agent), in combination with one or more pharmaceutically acceptable carriers (e.g., excipients). In making the compositions provided herein, the active ingredient is typically mixed with an excipient, diluted by an excipient or enclosed within such a carrier in the form of, for example, a capsule, tablet, or other container. When the excipient serves as a diluent, it can be a solid, semi-solid, or liquid material, which acts as a vehicle, carrier or medium for the active ingredient. Thus, the compositions can be in the form of tablets, pills, powders, suspensions, emulsions, solutions, syrups, aerosols (as a solid or in a liquid medium), soft and hard gelatin capsules, suppositories, sterile injectable solutions, and sterile packaged powders.

Some examples of suitable excipients include, without limitation, lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose. The formulations can additionally include, without limitation, lubricating agents such as talc, magnesium stearate, and mineral oil;

wetting agents; emulsifying and suspending agents; preserving agents such as methyl-and propylhydroxy-benzoates; sweetening agents; flavoring agents, or combinations thereof.

The nanocarriers (e.g., nanocarriers comprising one or more therapeutic agents) can be effective over a wide dosage range and are generally administered in a pharmaceutically effective amount. It will be understood, however, that the amount of the nanocarrier (e.g., nanocarriers comprising one or more therapeutic agents) actually administered will usually be determined by a physician, according to the relevant circumstances, including the condition to be treated, the chosen route of administration, the actual compound administered, the age, weight, and response of the individual subject, the severity of the subject's symptoms, and the like.

### EXAMPLES

The following examples are offered for illustrative purposes, and are not intended to limit the invention.

Materials: Epsilon-Polylysine (ϵ-poly-L-lysine, EPL; MW ˜4,700) was kindly supplied by Wako Chemical (Yokohama, Japan) or Wilshire Tech Inc. (Princeton, N.J.). β-cyclodextrin (β-CD), Dess-Martin periodinane (DMP), sodium triacetoxyborohydride, succinic anhydride, dipyrrolidino(N-succinimidyloxy)carbenium hexafluorophosphate (HSPyU), acetic anhydride, bovine serum albumin (BSA), diisopropylethylamine (DIEA), 1-adamantylamine (AD), ninhydrin, acetone, and ethanol were purchased from Fisher Scientific (Pittsburgh, Pa.), Sigma-Aldrich (Saint Louis, Mo.), or Acros Organics (Morris Plains, N.J.).

### Example 1

### Synthesis of Nanocarrier 4

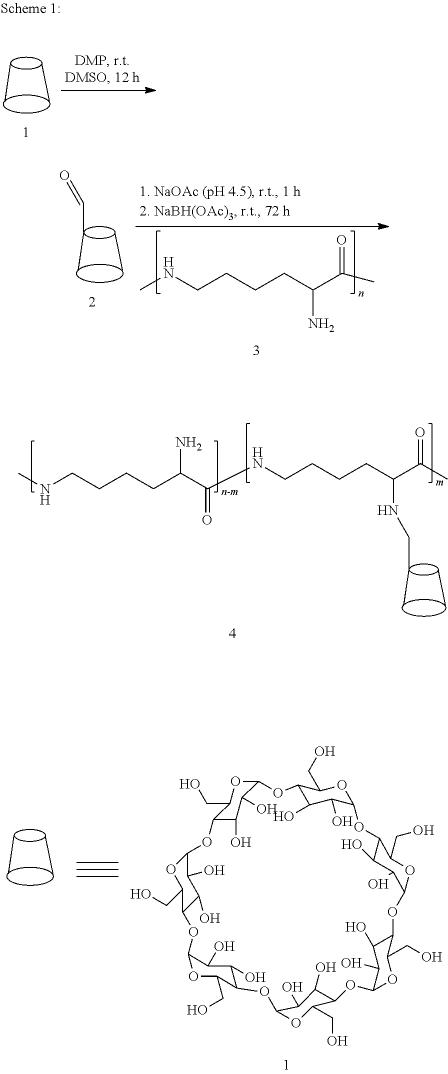

### Preparation of Nanocarrier 4

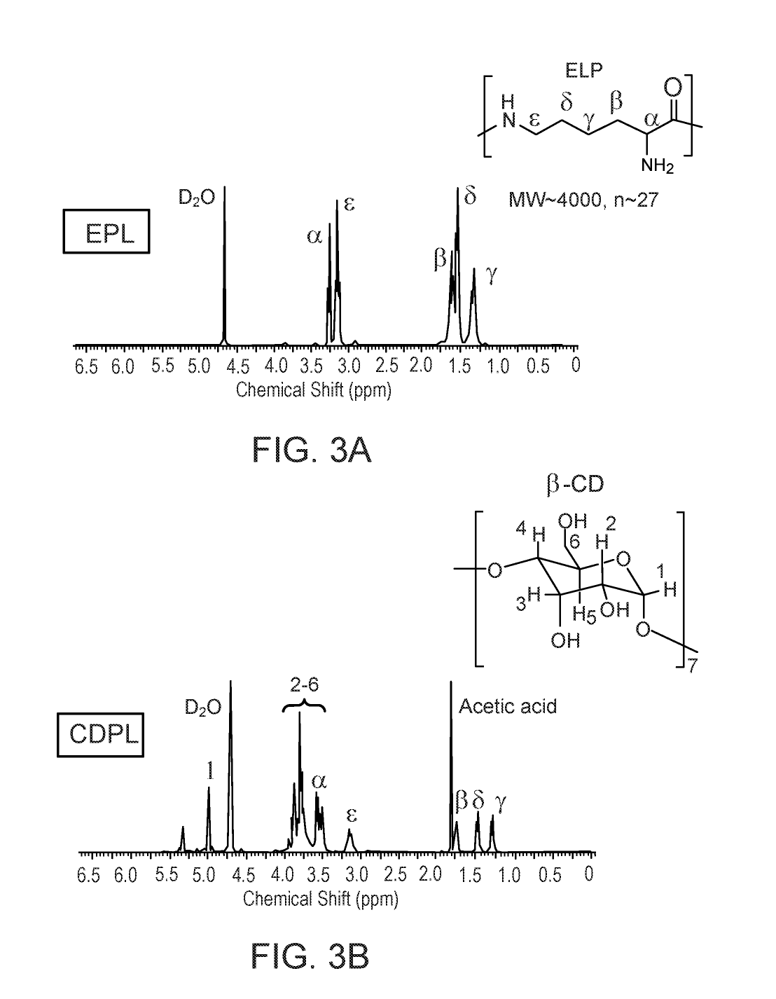

To prepare aldehyde β-CD 2 (Ald-CD), 1 g of β-CD 1 (0.88 mmol) and 0.8 g of DMP (1.9 mmol) were dissolved in anhydrous DMSO (25 mL) and stirred at room temperature. After overnight stirring, the solution was poured into 250 mL of cold acetone and kept at −20 ° C. for 2 h. The precipitate was retrieved by filtration and dissolved again in 20 mL of deionized water. The aqueous solution was then concentrated and recrystallized with cold acetone, and this operation was repeated twice to remove insoluble impurities. Finally, the white solid was recovered and dried in vacuo. A 700 mg (0.61 mmol) of Ald-CD 2 was dissolved in 25 mL of acetate buffer (0.2 M, pH 4.5) and then mixed with 100 mg of ϵ-polylysine (EPL) 3. After stirring for 1 h, 263 mg (1.2 mmol) of sodium triacetoxyborohydride was added into above reaction mixture. The mixture was additionally stirred for 72 h, and then neutralized by addition of a potassium carbonate aqueous solution (2 M). Dialysis was carried out in cellulose membrane with a molecular weight cutoff (MWCO) of 12-14 kDa for 24 h against DIW, the resulting solution was freeze-dried. The chemical composition of β-CD-conjugated EPL (CDPL) 4 was confirmed by ¹H NMR (see FIGS. 3A and 3B), where peak integration values of γ-position protons of polylysine at 1.3 ppm and 1-position protons of β-CD at 5 ppm were used to calculate the number of grafted β-CD on the EPL chain, which was determined to be an average of 6.7.

### Example 2

### Synthesis of Nanocarriers 6, 7, 8, and 9

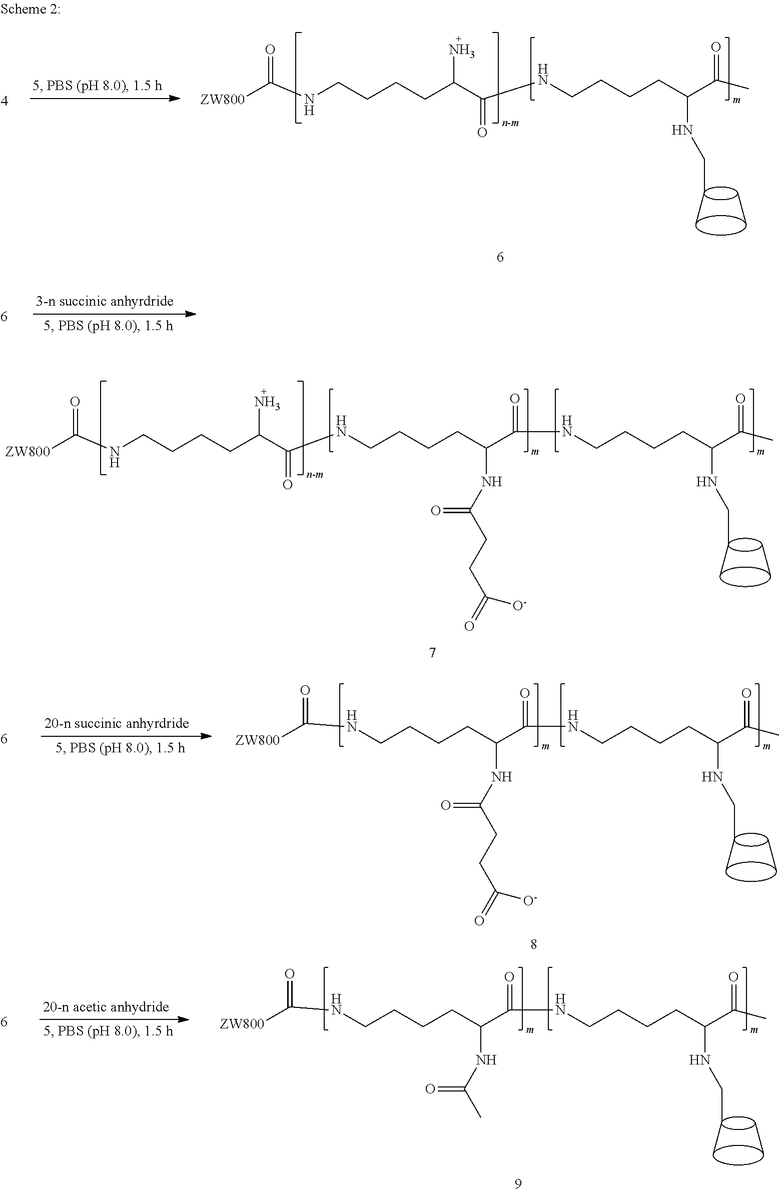

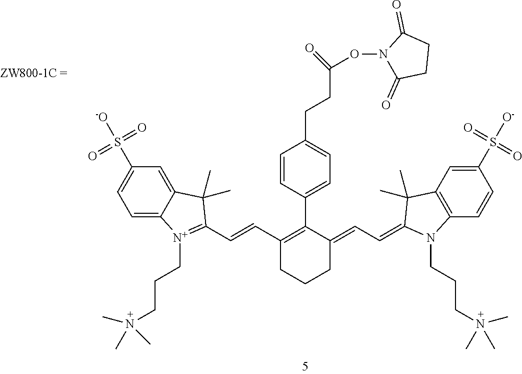

### Preparation of Activated Dye 5

ZW800-1C (500 mg, 0.5 mmol) was dissolved in 50 mL of anhydrous DMSO. Then, 0.5 mL of N,N-diisopropylethylamine (DIEA) and dipyrrolidino(N-succinimidyloxy)carbenium hexafluorophosphate (HSPyU; 410 mg, 1 mmol) were added to the solution. After stirring for 2 h at room temperature, the reaction mixture was poured in 250 mL of acetone/ethanol (1:1 v/v). The precipitate was filtered and washed with acetone/ethanol several times to remove excess reagents. The resulting ZW800-1C NHS ester 5 was dried overnight in vacuo.

### Preparation of Nanocarrier 6 (ZW800-CDPL⁺)

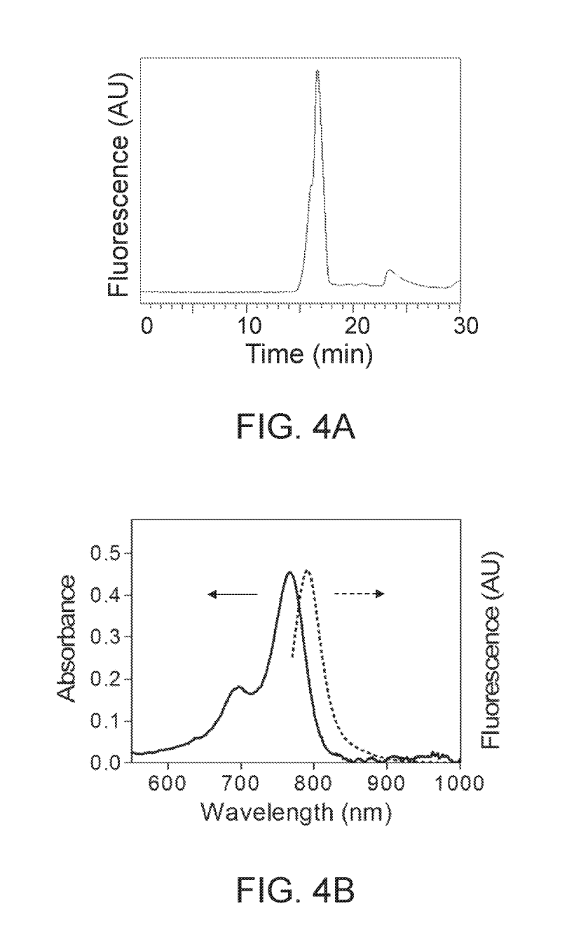

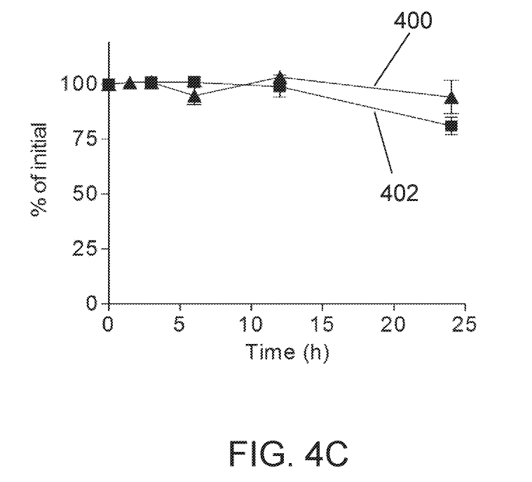

ZW800-1C-NHS ester 5 (50 μmol) was added to CDPL 4 (400 mg, 25 μmol) in 5 mL of PBS (pH 8.0). The reaction mixture was stirred for 12 h, then excess reagents were removed by Vivaspin centrifugal filters (10 kDa MWCO; Sartorious, New York, N.Y.). The resulting filtrate was lyophilized to yield nanocarrier 6, including positive charges. The size exclusion chromatogram of ZW800-CDPL⁺ 6 (FIG. 4A; first peak corresponds to 6, second peak corresponds to ZW800-1C) revealed successful conjugation of ZW800-1C 5 on the polymer backbone with >91% reaction yield. The absorbance (solid line) and fluorescence (dotted line) spectra (FIG. 4B) of ZW800-CDPL⁺ 6 (λ_{Abs}=769 nm; λ_{FL}=790 nm) represent no spectral changes compared with the control ZW800-1C. Next, serum stability was confirmed by incubating ZW800-CDPLs with fetal bovine serum (FBS; 5 w/v % in saline) at 37° C. (FIG. 4C; 400 is fluorescence, 402 is absorbance). As a result, the intensities of absorption and fluorescence decreased slightly over 24 h post-incubation (>81%), representing the stability of ZW800-CDPLs in the body without optical and physicochemical degradation.

### Preparation of Nanocarrier 7 (ZW800-CDP^{±})

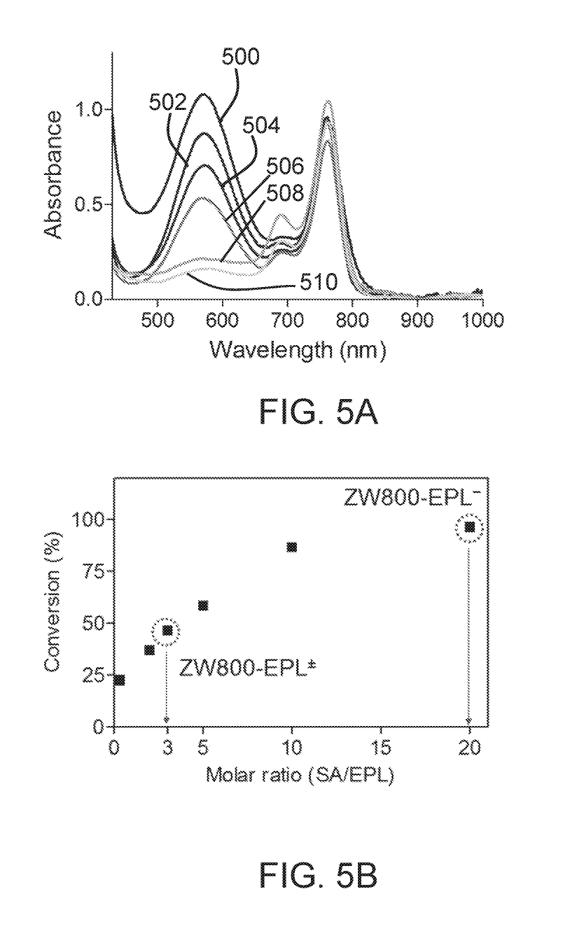

To prepare a zwitterionic nanocarrier 7 (both positive and negative charges) and a negatively charged nanocarrier 8, nanocarrier 6 was reacted with succinic anhydride (SA) to install pendant carboxylate groups on the lysine amino groups. To determine the ideal molar ratios of SA:nanocarrier 6 in order to prepare nanocarriers 7 and 8, succinic anhydride and nanocarrier 6 in pH 8.0 phosphate buffer solution (PBS) were reacted in molar ratios of SA to individual lysine units in the EPL of 0.33:1, 2:1, 3:1, 5:1, 10:1, and 20:1. FIG. 5A shows a plot of superimposed absorption spectra of ninhydrin reacted with unmodified primary amine groups in the polymer chain at each molar ratio (500, 0.33:1; 502, 2:1; 504, 3:1; 506, 5:1; 508, 10:1; 510, 20:1). FIG. 5B depicts a plot of percent conversion of succinylation (i.e., number of free amino groups consumed; this is obtained from the ninhydrin test discussed below) vs. molar ratio of succinic anhydride to each lysine unit. 50% succinylation of the amino groups occurred when the SA:EPL unit ratio was 3:1 (resulting in a zwitterionic nanocarrier with an equal number of positive and negative charges) and 100% conversion occurred when the SA:EPL unit ratio was 20:1 (resulting in an exclusively negatively charged nanocarrier). These ratios were used below in the preparation of zwitterionic nanocarrier 7 and negatively charged nanocarrier 8. The latter ratio was also used in preparing nanocarrier 9, wherein the free amino groups of the lysine units were capped with acetic anhydride rather than succinic anhydride, resulting in uncharged moieties.

To prepare nanocarrier 7, ZW800-1C-NHS ester 5 (50 μmol) was added to CDPL 4 (400 mg, 25 μmol) in 5 mL of PBS (pH 8.0). The reaction mixture was stirred for 12 h, then succinic anhydride (SA; 3 n μmol, where n is the number of lysine units in the EPL) was added to the reaction mixture and the mixture was vortexed for 1.5 h at room temperature. The solution was precipitated by adding acetone (14 mL) and washed with acetone five times followed by centrifugation to remove excess reagents. The resulting filtrate was lyophilized to yield nanocarrier 7, having both positive and negative charges.

### Preparation of Nanocarrier 8 (ZW800-CDPL⁻)

ZW800-1C-NHS ester 5 (50 μmol) was added to CDPL 4 (400 mg, 25 μmol) in 5 mL of PBS (pH 8.0). Succinic anhydride (20 n μmol, where n is the number of lysine units in the EPL) was added to the reaction mixture and the mixture was vortexed for 1.5 h at room temperature, then stirred for 12 h. Excess reagents were removed by Vivaspin centrifugal filters (10 kDa MWCO; Sartorious, New York, N.Y.). The resulting filtrate was lyophilized to yield negatively charged nanocarrier 8.

### Preparation of Nanocarrier 9 (ZW800-CDPL^{Ac})

ZW800-1C-NHS ester 5 (50 μmol) was added to CDPL 4 (400 mg, 25 μmol) in 5 mL of PBS (pH 8.0). Acetic anhydride (AA; 20 n μmol, where n is the number of lysine units in the EPL) was added to the reaction mixture and the mixture was vortexed for 1.5 h at room temperature, then stirred for 12 h. Excess reagents were removed by Vivaspin centrifugal filters (10 kDa MWCO; Sartorious, New York, N.Y.). The resulting filtrate was lyophilized to yield uncharged nanocarrier 9.

Purification and analysis: The purity of all nanocarriers was measured using size-exclusion chromatography (SEC) analysis on the Agilent HPLC system consisting of a 1260 binary pump with a 1260 ALS injector, a 35900E Photodiode Array detector (Agilent, 200-800 nm), and a 2475 multi-wavelength fluorescence detector (Waters, Ex 770 nm and Em 790 nm). A portion of the eluent flowed into the PDA equipped with an Ultrahydrogel 2000 (7.8×300 mm) SEC column. Mobile phase was 0.1% formic acid in water for 30 min with a flow rate of 0.75 mL/min.

### Example 3

### Measurement of Physicochemical Properties of Nanocarriers 6, 7, 8, and 9

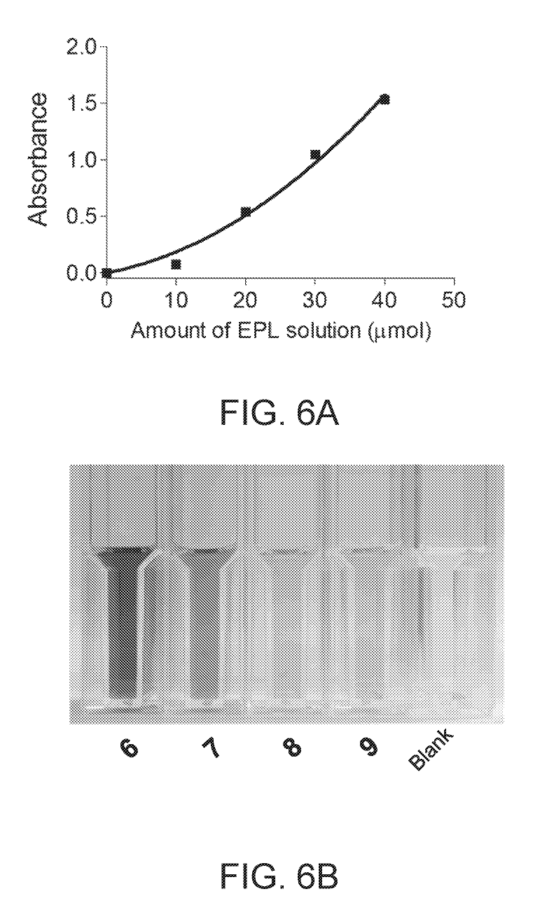

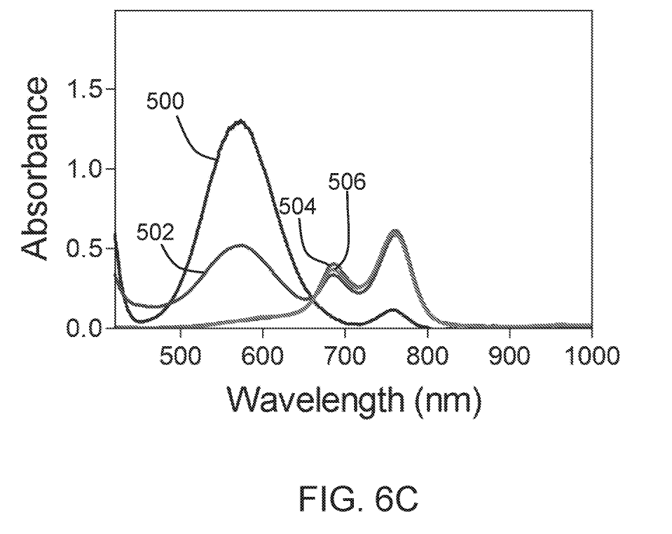

Ninhydrin test for estimation of the number of amine groups: Different volumes (0-20 μL) of standard amine-containing solutions (2 mM of EPL in water) for the standard calibration curve (FIG. 6A) and 20 μL of CDPLs 2 mM were added into test tubes. 1 mL of ninhydrin solution (8 wt % in ethanol) was then added to the prepared test tubes containing EPL, followed by placing them in a boiling water bath for 5 min and then cooling down in cold water. Then, the test tubes were filled with additional DIW up to 3 mL. FIG. 6B depicts a photograph of each nanocarrier and a blank. The absorbance changes of each solution at 570 nm were measured using a UV/Vis/NIR spectrometer (USB2000, Ocean Optics, Dunedin, Fla.), and the plots of absorbance vs. wavelength (nm) are depicted in FIG. 6C (plot 500 corresponds to nanocarrier 6; plot 502 corresponds to nanocarrier 7; plot 504 corresponds to nanocarrier 8; plot 506 corresponds to nanocarrier 9). FIG. 6C shows a peak at about 570 nm for nanocarriers 6 and 7 corresponding to free amino groups (with 7 showing a lower magnitude of absorbance due to the lower number of free amino groups). The nanocarriers with no free amines (8 and 9) show no peak in this region. The number of amino groups enabled calculation of the number of free ammonium and carboxylate groups and therefore the average number of charges each nanocarrier had (see row 2, Table 1).

Fluorescence correlation spectroscopy (FCS): The HD of ZW800-CDPLs 6-9 was calculated by both fluorescence correlation spectroscopy (FCS) and intrinsic viscosity-based approximation. Absorbance spectra of rhodamine-grafted CDPL solutions were measured using an Evolution 201 UV-Vis spectrophotometer (Thermo-Scientific) to determine their concentrations. 10 nM solutions were prepared, and 20 μL of droplets were placed in an 8 wells Lab-Tek borosilicate slide. FCS measurements were then performed on a single photon CLSM-FCS confocal microscope system (Confocor 2, Zeiss, Jena, Germany) using a 40× water immersion objective lens (C-Apochromat, 1.2 NA, Zeiss) and high sensitivity avalanche photodiodes. Calibration was performed using an aqueous solution of rhodamine 6G, and a laser excitation of 550 nm was used. As shown in row 4 of Table 1, all ZW800-CDPLs were smaller than 5.5 nm, indicating potential and preferable renal clearance.

Protein binding assay: To determine the changes in hydrodynamic diameter (HD) after serum protein binding, rhodamine-grafted CDPLs were first incubated in serum-containing media at 37° C. for 4 h. The amount of nonspecific serum protein binding to CDPL was then measured by loading each serum mixture on Bio-Gel P60 polyacrylamide gel (Bio-rad), and their retention times were measured using gel filtration chromatography (GFC; GE Akta GFC Purifier) with 1× PBS as an eluent. Absorbance spectra at 550 nm were measured to calculate the percentage of protein binding. Serum-free CDPLs and FBS alone were used as control, and the shifted peak was calculated by comparing the area under the curve between the original versus shifted. Percentage protein binding is shown in row 6 of Table 1, showing that the zwitterionic nanocarriers had the lowest extent of binding.

TABLE 1
Physicochemical properties of Nanocarriers 6-9 with charge variations.

| Physicochemical | | | | |
|---|---|---|---|---|
| property | 6 | 7 | 8 | 9 |
| Net charges | Positive | Zwitterionic | Negative | No charge |
| # Charges (NH₃⁺/COO⁻) | 24/0 | 12/12 | 0/23 | 0/0 |
| Average MW (g/mol) | 16,000 | 16,900 | 17,700 | 16,700 |
| HD [nm], measurement | 4.4 | 4.6 | 4.9 | 4.8 |
| HD [nm], theory | 4.6 | 4.7 | 4.9 | 4.7 |
| Protein binding (%) | 23 | 14 | 26 | 25 |

### Example 4

### Measurement of Pharmacokinetic Properties of Nanocarriers 6, 7, 8, and 9

### General Methods

Stability and pH-responsive drug release tests: Each ZW800-CDPL (2 mM in water; 20 μL) was mixed with Cy3-imatinib solution (10 mM in DMSO/PBS, 50/50 v/v %; 40 μL) and the mixture was vortexed for 24 h at room temperature. Then, the mixture was loaded on micro Bio-Spin P-6 gel columns (Bio-rad) to remove unbound excess imatinib. To monitor the release of imatinib at different pH environments, Cy3-imatinib loaded ZW800-CDPL was dispersed in PBS (pH 5.0, 1 mL) and PBS (pH 7.4, 1 mL), respectively. 10-50 μL of the imatinib-CDPL solutions were taken at each time point, and released imatinib was removed by the micro Bio-Spin P-6 gel column. The absorbance of filtered CDPL solutions was then measured at 550 nm to calculate the amount of imatinib released. The percentage of the released imatinib was calculated by the following formula, [(Absᵢₙᵢₜᵢₐₗ−Abs_{time point})/Absᵢₙᵢₜᵢₐₗ×100].

In vivo biodistribution and pharmacokinetics of nanocarriers: Animals were housed in an Association for Assessment and Accreditation of Laboratory Animal Care (AAALAC)-certified facility and were studied under the supervision of the Institutional Animal Care and Use Committee (IACUC) of Beth Israel Deaconess Medical Center (BIDMC) in accordance with the approved institutional protocol (# 057-2014). Six weeks old CD-1 mice (male; 25-30 g) were purchased from Charles River Laboratories (Wilmington, Mass.). Mice were maintained under anesthesia by intraperitoneal injection with 100 mg/kg ketamine and 10 mg/kg xylazine (Webster Veterinary, Fort Devens, MA) for the entire duration of the experiment. The end of the tail was cut to enable blood extraction. Before injection, blood was then sampled in heparinized capillary tubes (Fisher Scientific, Pittsburgh, Pa.) as a reference and collected blood was stored in an ice box to prevent clotting. Mice were injected with 10 nmol of each ZW800-CDPL in saline containing 5 wt/v % BSA and blood was sampled at the following time points (1, 3, 5, 10, 30, 60, 120, 180, and 240 min) to estimate distribution (t_{1/2a}) and elimination (t_{1/2β}) blood half-life values. Mice were imaged using the in-house built real-time intraoperative MR imaging system. A 760 nm excitation laser source (4 mW/cm) was used with white light (400-650 nm; 40,000 lux). Color and MR fluorescence images were acquired simultaneously with customized software at rates of up to 15 Hz over a field of view with 15 cm in diameter. After 4 h post-injection, mice were sacrificed to image organs and collected urine from the bladder. At least 3 mice were analyzed for each sample.

In vivo tumor targeting and drug delivery: To establish tumor-xenografted nude mice, gastrointestinal tumor (GIST) cells were cultured in Dulbecco's Modified Eagle Medium (DMEM) with 5% FBS and 100 units/ml of penicillin and streptomycin. NCr nu/nu mice (Taconic Farms, Germantown, N.Y.) were inoculated subcutaneous injection with 2×10⁶ GIST cells suspended in 150 μL of saline/matrigel (50 v/v %) at the left flank. Once the tumor reached a size of 0.5 cm, 40 nmol of Cy3-loaded ZW800-CDPL^{±} in saline containing 5% BSA was injected through tail vein. Tumor mice were imaged using a real-time intraoperative MR imaging system at the following time points (10, 30, 60, 120, 180, 240, 720 and 1440 min) and then scarified for ex vivo imaging and histological evaluations. For histology, fluorescence microscopy was performed on a Nikon 1E2000 with two custom filter sets (Chroma Technology, Brattleboro, Vt., USA).

Quantitative analysis: The fluorescence and background intensities of a region of interest over each tissue were quantified using customized imaging software and ImageJ v1.48 (National Institutes of Health, Bethesda, Md.). The signal-to-background ratio (SBR) was calculated as SBR=fluorescence/background, where background is the fluorescence intensity of muscle. A one-way ANOVA followed by Tukey's multiple comparisons test was used to assess the statistical difference. P value of less than 0.05 was considered significant: *P<0.05, **P<0.01, and ***P<0.001. Results are presented as mean±standard deviation (s.d.).

### Results/Discussion

The collected blood samples were centrifuged for 20 min at 3000 rpm in order to separate serum and blood plasma, and supernatants were then filled into capillary microtubes. Fluorescence intensities of the microtubes were measured using the in-house built MR imaging system. Results were presented as a bi-exponential decay curve using Prism version 4.0a software (GraphPad, San Diego, Calif.).

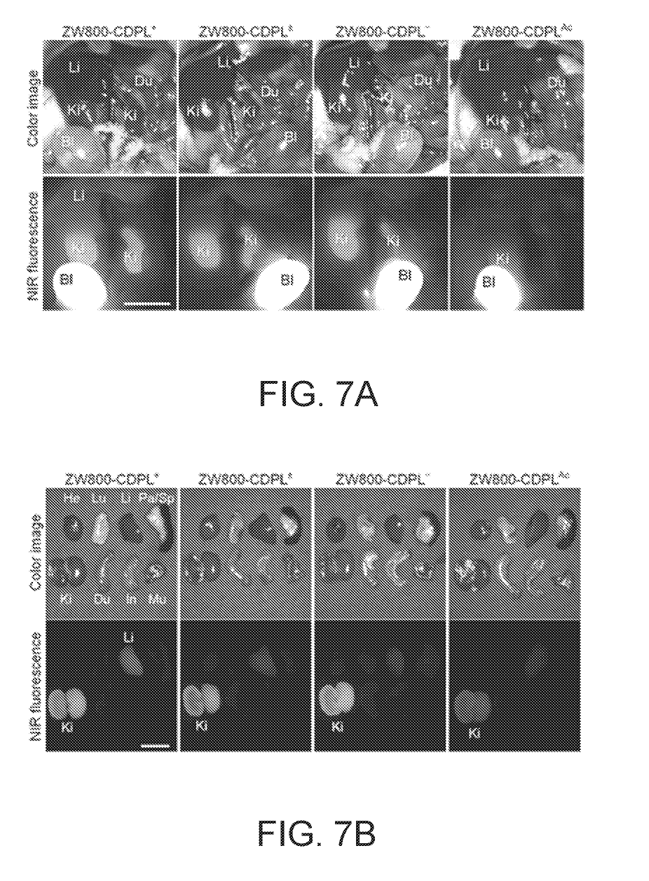

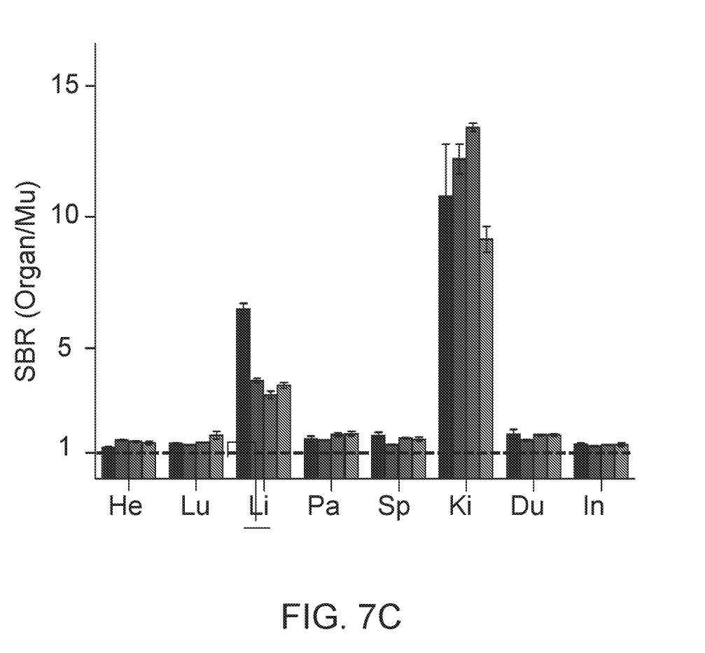

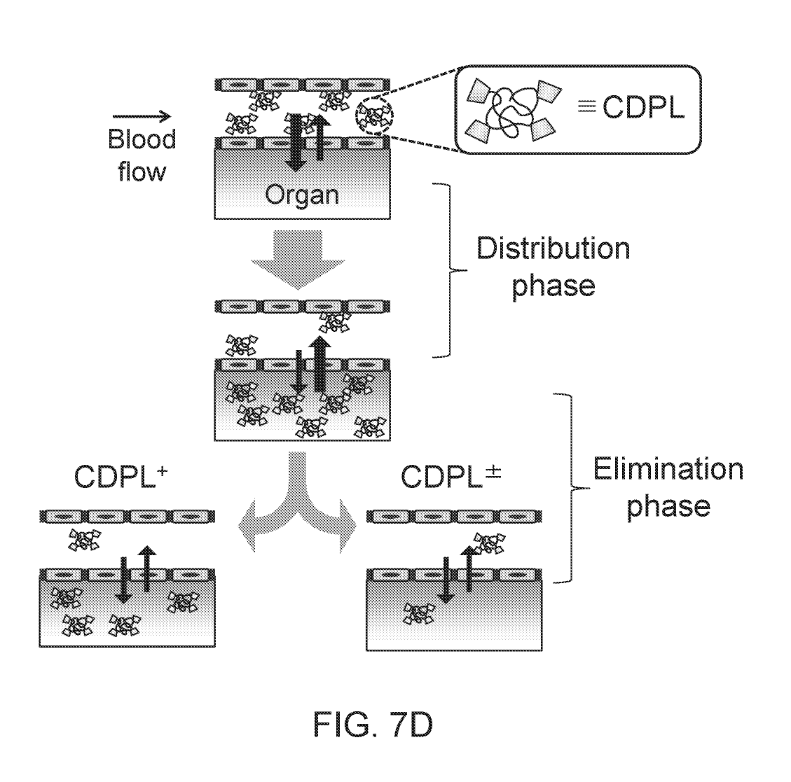

The biodistribution, renal clearance, and pharmacokinetics of 6, 7, 8, and 9 in CD-1 mice were investigated. The initial distribution was continuously observed for 1 min by the real-time imaging system immediately after a single intravenous injection of each nanocarrier. Overall, the nanocarriers distributed rapidly in the blood, heart, lung, liver, and other major organs within 1 min post-injection, and then gradually accumulated into kidneys, followed by renal excretion to the bladder. The MR fluorescence signals of the nanocarriers were mainly located in the urinary system 4 h post-injection (FIG. 7A). Interestingly, nanocarrier 6 showed relatively high fluorescence in the liver and abdominal cavity because of electrostatic interactions with the negatively charged cell membrane in each. In contrast, all the other nanocarriers left no fluorescence signals in the liver, of which signal-to-background ratio (SBR; organs vs. muscle) was calculated in FIG. 7A and 7B along with other resected organs, and a bar graph of the relative SBR for each nanocarrier in each organ is shown in FIG. 7C (first set of bars, nanocarrier 6; second set of bars, nanocarrier 7; third set of bars, nanocarrier 8; fourth set of bars, nanocarrier 9). These results indicate that zwitterionic, negative, or acetylated CDPLs can elude nonspecific uptake by the reticuloendothelial system (RES) and exclusively excrete (>80% ID) to the bladder within 4 h post-injection (FIG. 7D).

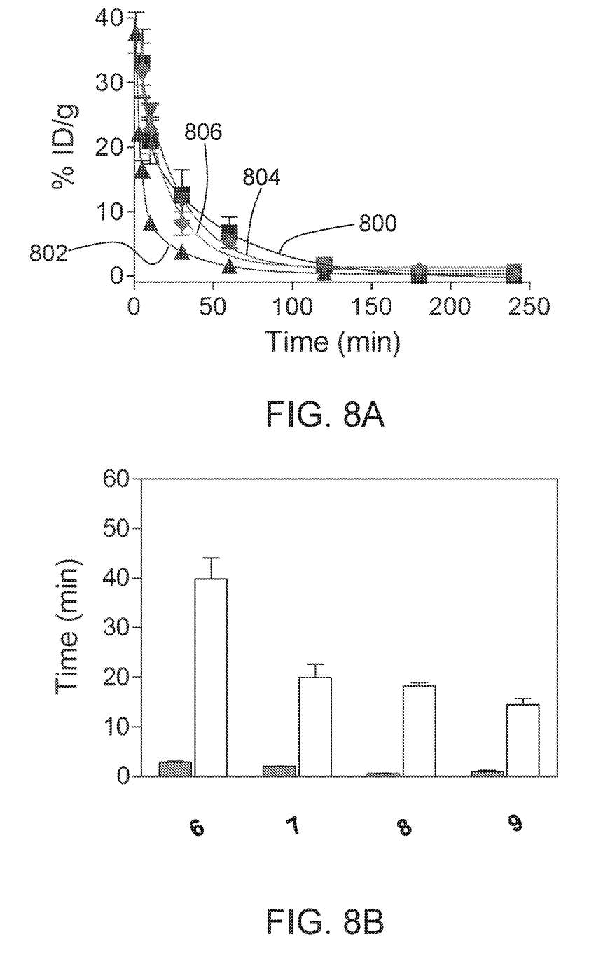

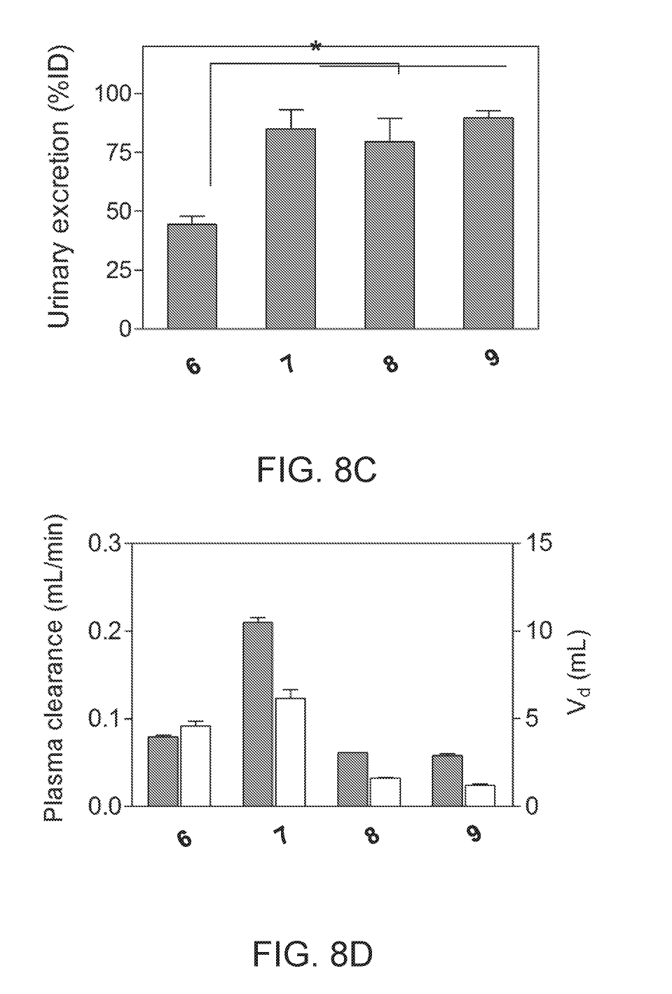

The pharmacokinetic parameters of ZW800-CDPLs after a single intravenous injection were summarized in Table 2. The blood concentration curves represent that the nanocarriers exhibit a two-compartment profile of in vivo kinetics (FIG. 8A; plot 800 corresponds to nanocarrier 6, plot 802 corresponds to nanocarrier 7, plot 804 corresponds to nanocarrier 8, plot 806 corresponds to nanocarrier 9). The rapid initial decay of blood concentration was reflected by the efficient initial distribution into capillaries, and the final concentrations after 4 h post-injection reached close to 0% ID/g representing rapid elimination from the body by the systemic clearance. The half-life values of the nanocarriers (FIG. 8B; first (gray) bar for each nanocarrier corresponds to distribution half-life, second (white) bar for each nanocarrier corresponds to terminal half-life) range from 0.52±0.12 (nanocarrier 8) to 2.86±0.15 min (nanocarrier 6) during the distribution phase (t_{1/2α}), and from 14.41±1.25 (nanocarrier 9) to 39.80±4.17 (nanocarrier 6) for the terminal phase (t_{1/2β}). Among them, nanocarrier 6 showed relatively longer blood half-lives than the other nanocarriers (**P<0.01), which might result from the nonspecific interaction associated with plasma proteins. In addition, urinary excretion of non-positive charged nanocarriers was >80% ID at 4 h post-injection (FIG. 8C), while only approximately 45% ID of ZW800-CDPL⁺ was found in the bladder (**P<0.01). The blood clearance and urinary excretion of CDPLs are similar to those of renal clearable small molecule fluorophores such as ZW800-1, ZW800-1C, and ZW700-1, but much faster than those of previously reported renal clearable inorganic nanoparticles such as silica, gold cluster, and quantum dots. The values for plasma clearance and volume of distribution were estimated based on the pharmacokinetics data depicted in FIG. 8D (first (gray) bar for each nanocarrier corresponds to plasma clearance half-life, second (white) bar for each nanocarrier corresponds to volume of distribution). Despite the relatively short blood half-life, the plasma clearance value of zwitterionic ZW800-CDPL appeared to be 0.21 mL/min, which is 2.6-fold faster than that of negative or acetylated CDPLs. Interestingly, the volume of distribution for nanocarrier 7 also showed the highest value among the tested albeit no significant signals in the major organs except kidneys. To support these results, protein binding assay was carried out by incubating the nanocarriers in 5% FBS for 4 h, and gel filtration chromatography (GFC) was used to measure the changes in retention time. Consequently, the nanocarrier 7 exhibited only minimum adsorption with serum proteins (14%), while nanocarrier 6 and nanocarrier 8 resulted in 23% and 26% of protein binding, respectively (Table 1). This is explained by the pharmacokinetics data including plasma clearance and volume of distribution for CDPL derivatives: nanocarrier 7 systemically circulated and distributed to the whole body without nonspecific uptake by the RES, then eliminated efficiently from the body.

TABLE 2
Pharmacokinetic parameters for ZW800-CDPL derivatives.

| Pharmacokinetics | 6 | 7 | 8 | 9 |
|---|---|---|---|---|
| Injected dose, mole [nmol] | 10 | 10 | 10 | 10 |
| Injected dose, amount [μg] | 122 | 133 | 145 | 131 |
| t_{1/2} α [min] | 2.86 ± 0.15 | 1.97 ± 0.11 | 0.52 ± 0.12 | 0.99 ± 0.19 |
| t_{1/2} β [min] | 39.80 ± 4.17 | 19.87 ± 2.77 | 18.28 ± 0.61 | 14.41 ± 1.25 |
| Urinary excretion [% ID] | 44.55 ± 3.36 | 85.07 ± 8.18 | 79.65 ± 9.86 | 89.62 ± 3.07 |
| AUC [% ID · min] | 1252 | 465 | 1644 | 1214 |
| Plasma clearance [mL/min] | 0.079 | 0.21 | 0.061 | 0.082 |
| Volume of distribution [mL] | 4.58 | 6.16 | 1.60 | 1.71 |

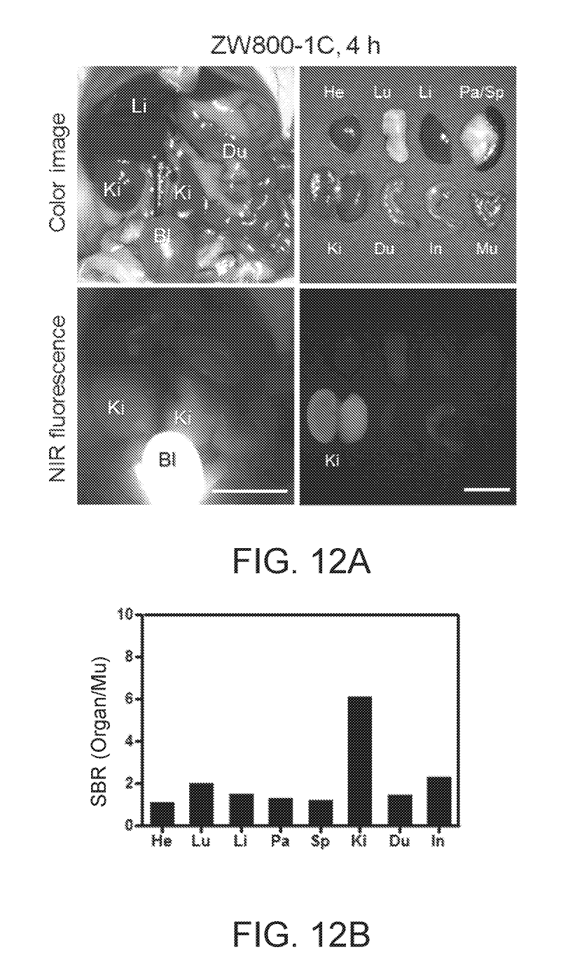

To demonstrate the in vivo biodistribution and clearance of ZW800-1C, as a control experiment, 10 nmol of ZW800-1C was injected intravenously into CD-1 mice 4 h prior to imaging. FIG. 12A depicts the intraoperative imaging of the abdominal cavity and resected tissues/organs both photographically and using NIR fluorescence (abbreviations used are: Bl, bladder; Du, duodenum; He, heart; In, intestine; Ki, kidneys; Li, liver; Lu, lungs; Mu, muscle; Pa, pancreas; Sp, spleen). The MR fluorescence image shows that the unconjugated zwitterioinic dye excretes to the bladder 4 h after injection. FIG. 12B depicts a signal-to-background ratio (SBR) of each organ against muscle, showing the highest SBR in the kidneys. These results show that the intact unconjugated dye undergoes rapid renal clearance.

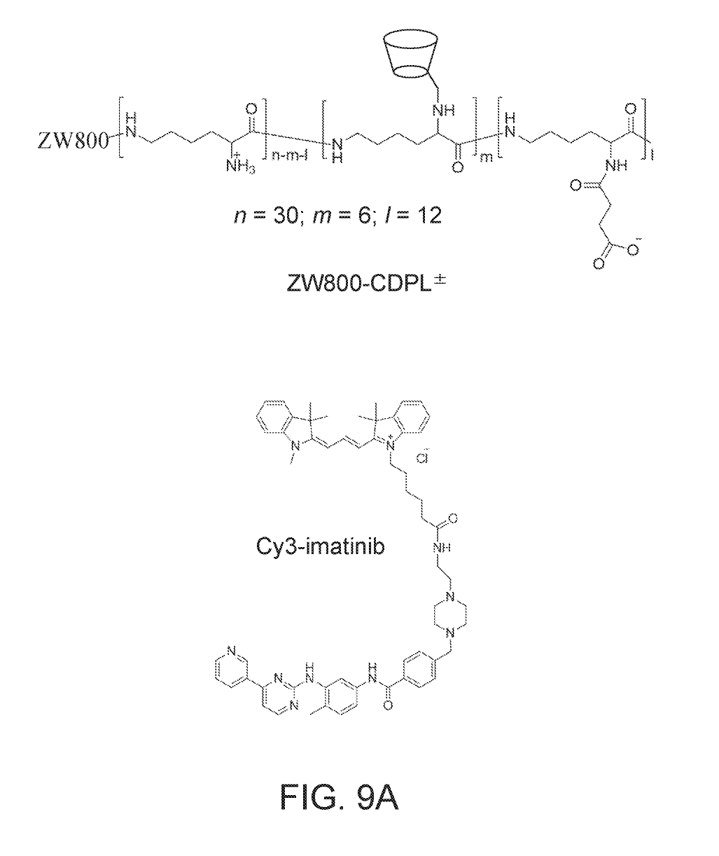

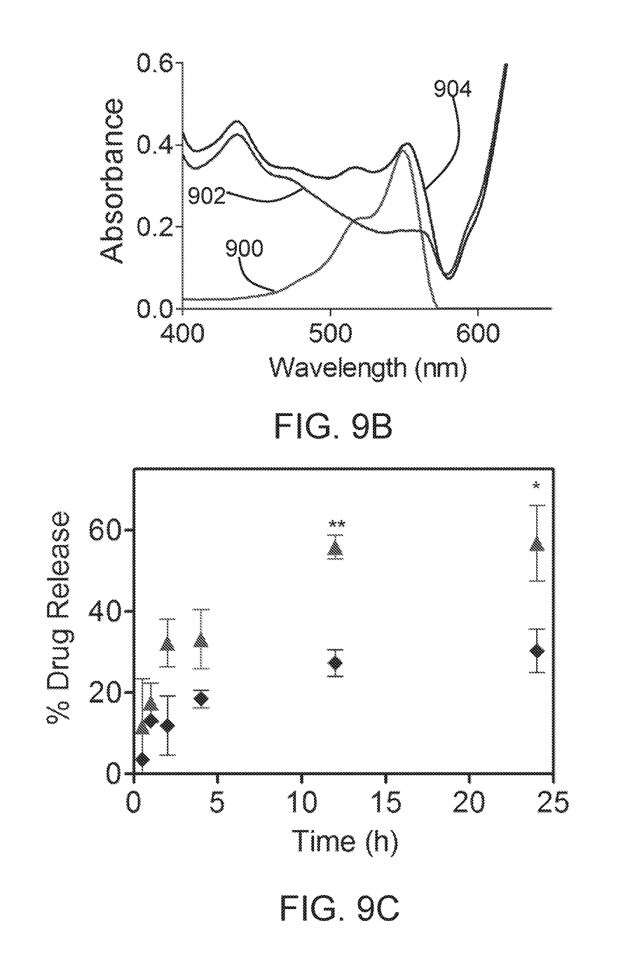

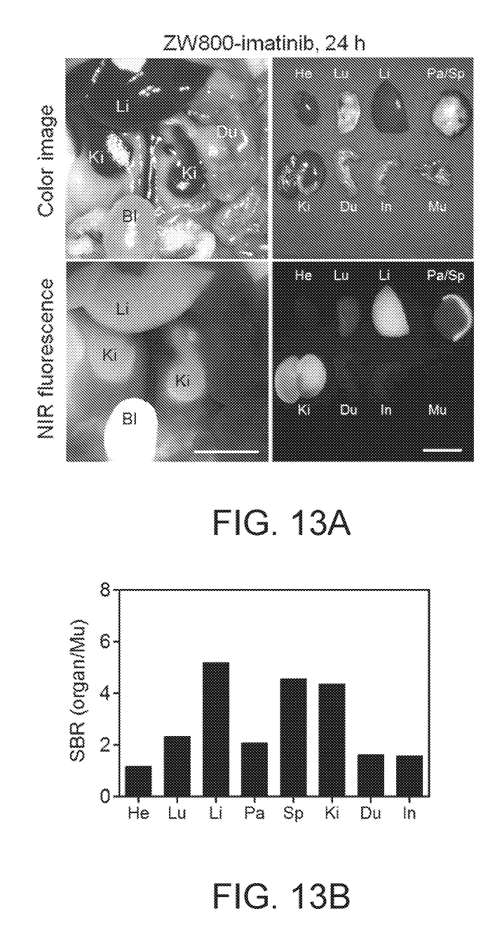

To demonstrate the in vivo biodistribution and clearance of ZW800-1C conjugated to imatinib (ZW800-imatinib), 10 nmol of the conjugate was injected intravenously into CD-1 mice 4 h prior to imaging. FIG. 13A depicts the intraoperative imaging of the abdominal cavity and resected tissues/organs both photographically and using MR fluorescence. The MR fluorescence image shows that the dye-imatinib conjugate distributes in multiple organs (e.g., liver, kidneys, spleen, bladder) with the bladder showing the highest signal. FIG. 13B depicts a signal-to-background ratio (SBR) of each organ against muscle, showing an organ distribution that is less selective than that of ZW800-1C because of the conjugated imatinib. To demonstrate efficient tumor targeting and drug delivery using nanocarrier 7, both xenograft and genetically engineered gastrointestinal stromal tumor (GIST) mouse models were additionally recruited. Imatinib, a tyrosine-kinase inhibitor for treating GIST, was selected as a therapeutic drug because it forms a 1:1 stoichiometric host-guest complex with β-CD. Imatinib was conjugated with a fluorescent dye, Cy3 NHS Ester (GE Healthcare), to track the distribution and clearance of imatinib in tumor-bearing mice. Nanocarrier 7 and the dye-conjugated imatinib are shown in FIG. 9A. Prior to carrying out in vivo tumor targeting, the imatinib-CDPL^{±} inclusion complex was tested for pH-induced drug release by measuring the changes in absorbance spectra of Cy3 (FIGS. 9B, 900 corresponds to Cy3-imatinib, 902 corresponds to ZW800-CDPL, 904 corresponds to inclusion complex; and 9C, triangles correspond to pH 5.0, diamonds correspond to pH 7.4). While imatinib-loaded ZW800-CDPL^{±} was relatively stable at pH 7.4, up to 60% of imatinib was released from the CDPL delivery vehicle in 12 h post-incubation at pH 5.0 due to the reduced hydrophobic interactions between imatinib and the apolar cavity of β-CD. This result suggests that the inclusion complex is stable at the physiological environment (pH 7.4), but releases the complexed drugs efficiently in the tumor microenvironment (pH 5.0).

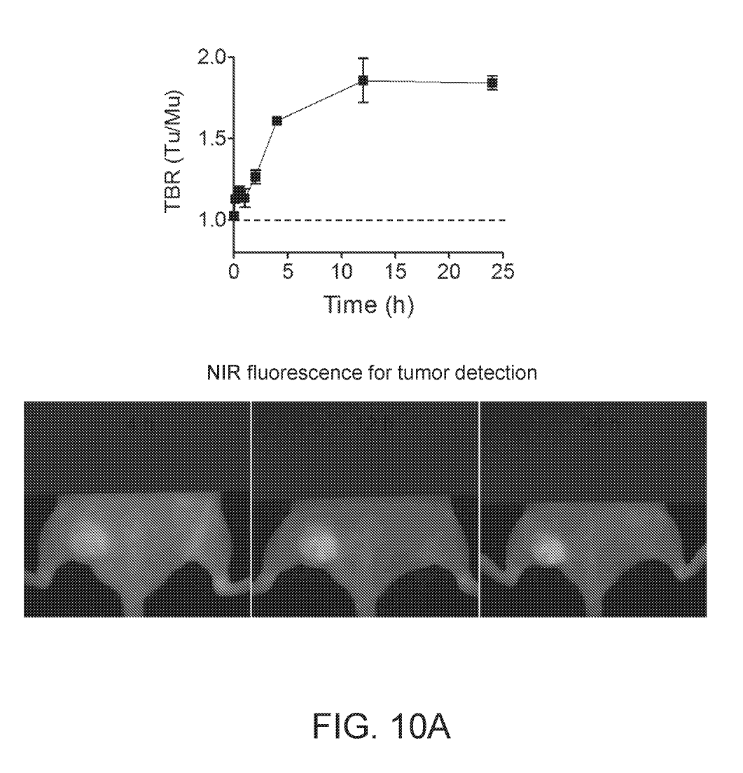

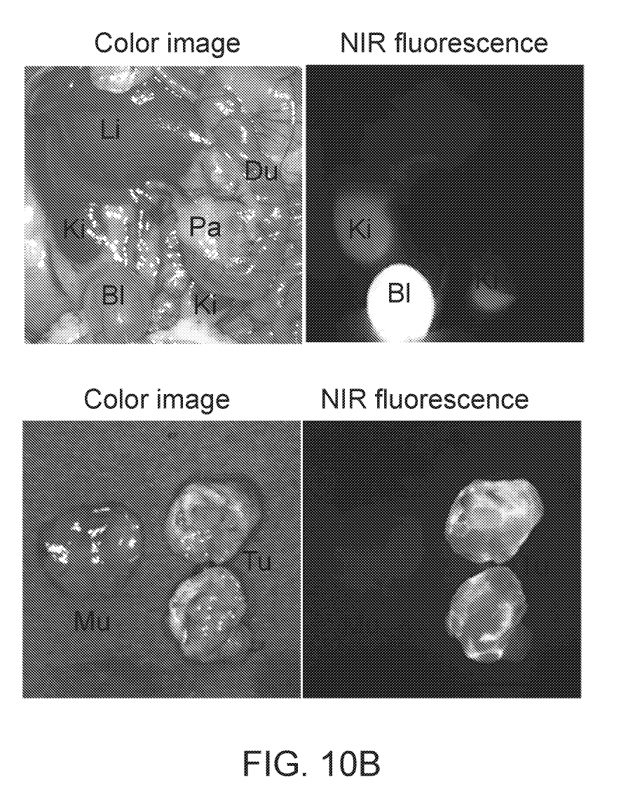

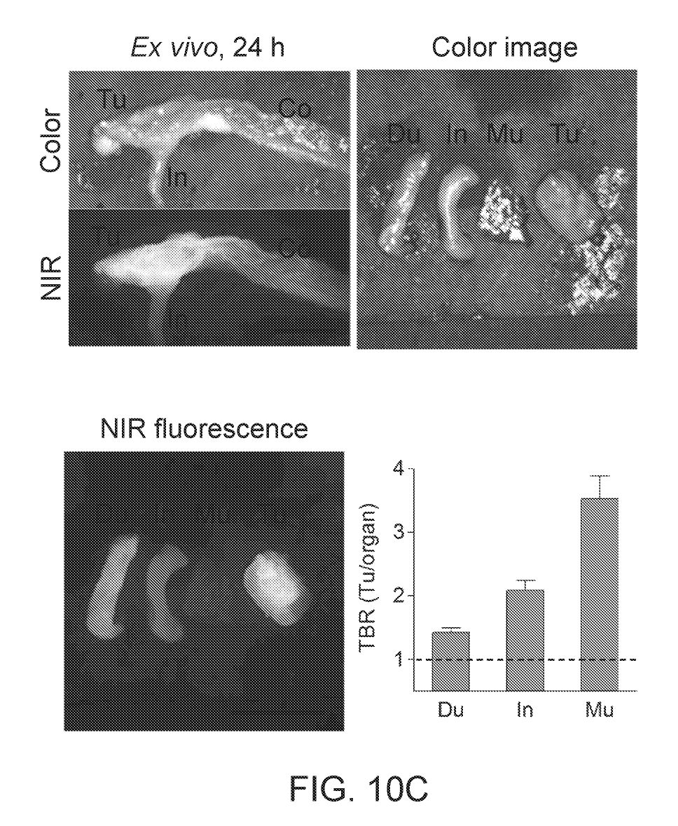

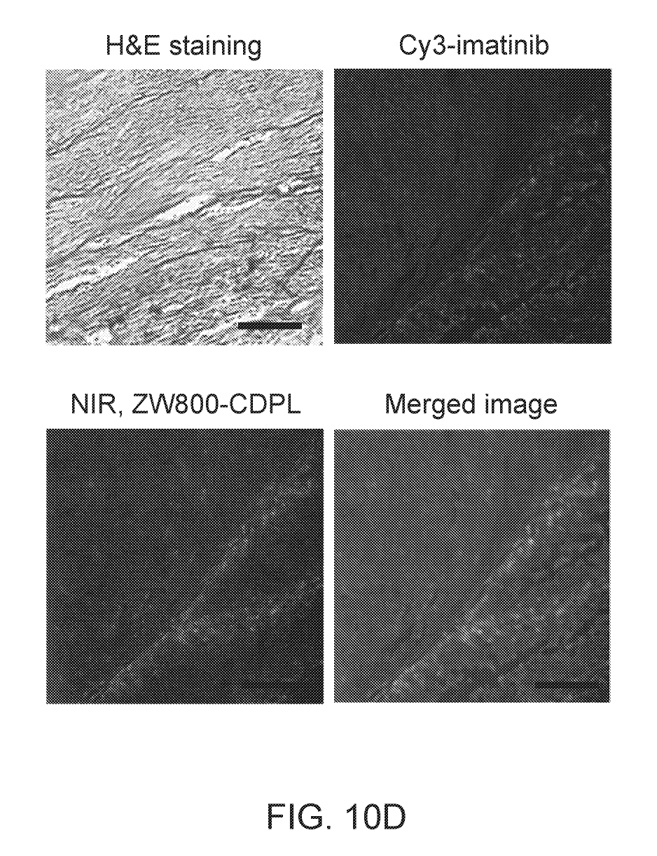

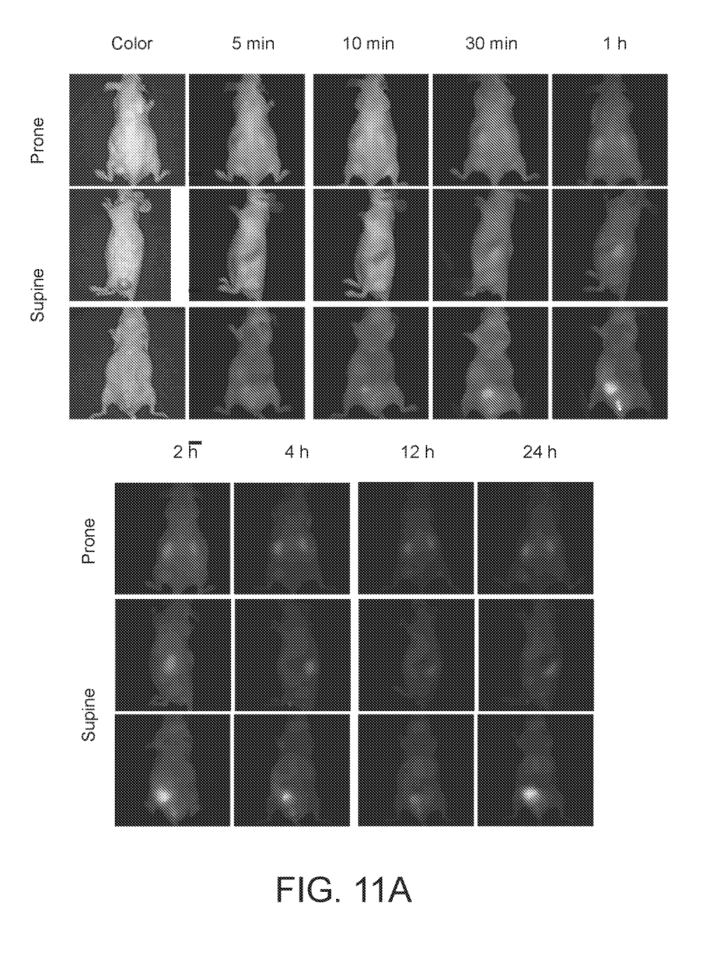

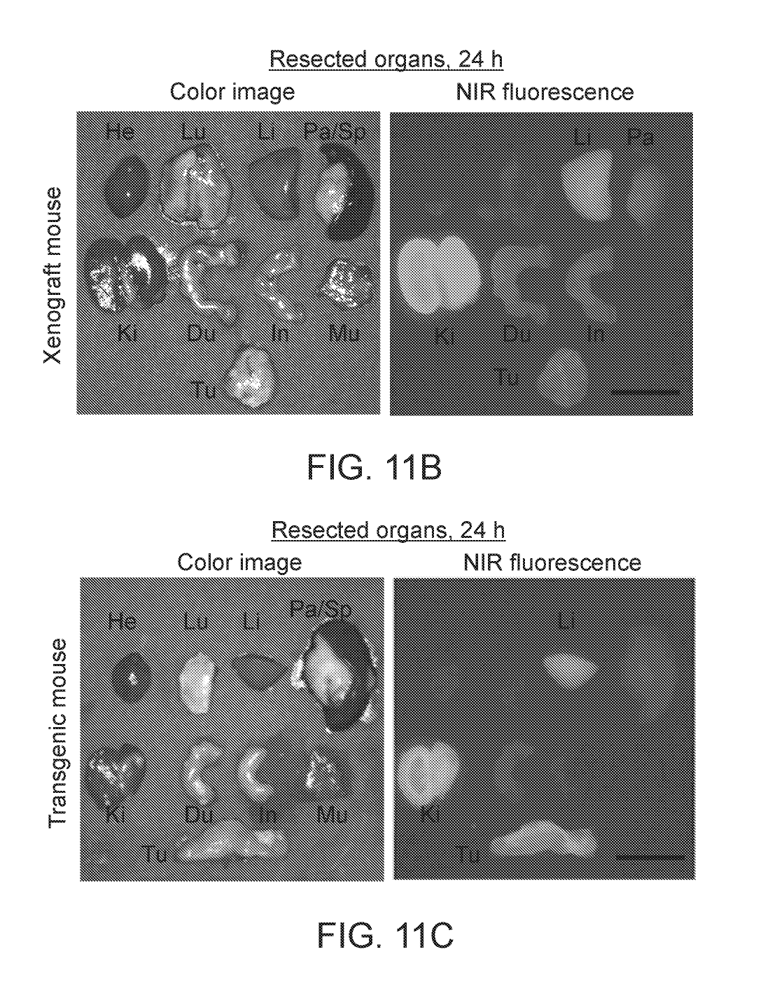

Next, the imatinib-CDPL^{±} complex was administered intravenously into GIST-bearing xenograft mice, and real-time intraoperative MR imaging was performed for 24 h post-injection (FIGS. 10A and 11A-11C). The tumor-to-background ratio (TBR) increased significantly over the time course of 12 h post-injection, and remained constant up to 24 h.

This result demonstrates that the imatinib-loaded CDPL^{±} successfully target the tumor region by the enhanced permeation and retention (EPR) effect. The xenograft mice were then sacrificed at 24 h post-injection and their abdominal cavity was observed to confirm biodistribution and clearance (FIG. 10B). Almost no background signal was observed in the major organs except the urinary excretory system including kidneys and bladder where

ZW800-CDPL^{±} is actively being eliminated. Tumors were resected subsequently along with other tissues and organs, and their MR fluorescence signal was compared against to muscle, of which TBR marked over 8.0 (FIG. 10B).

Furthermore, the drug delivery and tumor targeting efficiency of zwitterionic CDPL was demonstrated in genetically engineered GIST mice having tumors in the cecum area since birth. The imatinib-CDPL^{±} complex was injected intravenously into the GIST-bearing mice 24 h prior to imaging, and their tumors were imaged along with duodenum, intestine, and muscle along with their TBRs calculated for the duodenum, intestines, and muscle (FIG. 10C). The complex successfully targeted tumors around the cecum, but showed partial uptake in liver and pancreas. This is mainly because of the lipophilicity of imatinib, which is not fully compensated by the formation of inclusion complex with β-CD as well as by the zwitterionic property of CDPL (FIG. 11C). However, the imatinib-loaded CDPL avoided nonspecific uptake by lung and spleen, while the same dose of imatinib-conjugated with ZW800-1 showed relatively high nonspecific uptake by the RES because of interaction with macrophages. Tumors were then resected, and the intratumoral microdistribution of imatinib-loaded CDPL^{±} was investigated by H&E histology and fluorescence microscopy (FIG. 10D). Fluorescence images by red (upper right image, 590-650 nm) and MR (lower left image, 790-830 nm) filters were taken in order to detect Cy3-imatinib and ZW800-CDPL, respectively. Interestingly, ZW800-CDPL^{±} (pseudocolored in lime green) was predominantly observed in the boundary of tumoral regions and the signals of Cy3-imatinib (pseudocolored in red) were spread out intratumorally. This result indicates that zwitterionic CDPLs can successfully deliver hydrophobic drugs to the tumor site by forming a stable inclusion complex, and the anticancer drug is released from the delivery vehicle at the acidic pH generated by the tumor microenvironment.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A nanocarrier, comprising one or more cyclodextrin moieties conjugated to a polymer.

2. The nanocarrier of claim 1, wherein the polymer defines a micelle, a liposome, a nanosphere, a dendrimer, or a hollow shell.

3. The nanocarrier of claim 2, wherein the polymer comprises ϵ-polylysine, L-polylysine, polylactic acid, and poly(lactic-co-glycolic acid), polyaspartic acid, polyglutamic acid, or polyglutamic acid-poly(ethylene glycol) copolymer.

4. The nanocarrier of claim 1, wherein the cyclodextrin moiety is derived from α-cyclodextrin, β-cyclodextrin, γ-cyclodextrin, 2-hydroxypropyl-β-cyclodextrin, 2-hydroxypropyl-γ-cyclodextrin, methyl-β-cyclodextrin, a β-cyclodextrin thioether, or a cyanoethylated β-cyclodextrin.

5. The nanocarrier of claim 1, wherein at least one cyclodextrin moiety is conjugated to an amino group of the polymer.

6. (canceled)

7. The nanocarrier of claim 1, wherein the nanocarrier further comprises a contrast agent, wherein the contrast agent is conjugated to the polymer.

8. The nanocarrier of claim 7, wherein the contrast agent comprises a near-infrared fluorophore selected from the group consisting of ZW800-1C, ZW800-1, ZW800-3C, ZW700-1, indocyanine green (ICG), Cys, Cy5.5, Cy7, Cy7.5, IRDye800-CW (CW800), and ZWCC.

9. (canceled)

10. The nanocarrier of claim 1, wherein the nanocarrier comprises one or more therapeutic agents that form a complex with the one or more cyclodextrin moieties.

11. The nanocarrier of claim 10, wherein the one or more therapeutic agents comprise an anticancer agent selected from the group consisting of afatinib, AG 879, alectinib, altiratinib, apatinib, ARQ-087, ARRY-112, ARRY-523, ARRY-651, AUY-922, AXD7451, AZ-23, AZ623, AZ64, AZD4547, AZD6918, AZD7451, BGJ398, binimetinib, BLU6864, BLU9931, brivatinib, cabozantinib, CEP-751, CEP-701, cetuximab, CH5183284, crizotinib, CT327, dabrafenib, danusertib, DCC-2036, DCC-2157, dovitinib, DS-6051, encorafenib, erdafitinib, erlotinib, EWMD-2076, gefitinib, GNF-4256, GNF-5837, Gö 6976, GTx-186, GW441756, imatinib, K252a, lapatinib, lenvatinib, Loxo-101, Loxo-195, lucitanib, LY2874455, MGCD516, motesanib, nilotinib, nintedanib, NVP-AST487, ONO-5390556, orantinib, panitumumab, pazopanib, PD089828, PD166866, PD173074, pertuzumab, PF-477736, PHA-739358, PHA-848125AC, PLX7486, ponatinib, PZ-1, quercetin, regorafenib, RPI-1, ruxolitinib, RXDX101, RXDX105, semaxanib, sorafenib, SPP86, SSR128129E, SU4984, SU5402, SU6668, SUN11602, Sunitinib, TAS120, TG101209, TPX-0005, trastuzumab, TSR-011, vandetanib, vatalanib, VSR-902A, and XL-184.

12-16. (canceled)

17. The nanocarrier of claim 10, wherein the one or more therapeutic agents are conjugated with a fluorescent dye.

18-22. (canceled)

23. The nanocarrier of claim 10, wherein at least about 60% of the therapeutic agent is released from the nanocarrier at a pH of about 5.0.

24-32. (canceled)

33. The nanocarrier of claim 1, wherein the nanocarrier comprises one or more positively charged moieties and one or more negatively charged moieties.

34-38. (canceled)

39. The nanocarrier of claim 1, wherein the nanocarrier has no charged moieties.

40. The nanocarrier of claim 1, wherein the nanocarrier comprises an ammonium group or a carboxylate group.

41. (canceled)

42. The nanocarrier of claim 1, wherein the average molecular weight of the nanocarrier is from about 10,000 g/mol to about 22,000 g/mol.

43-44. (canceled)

45. The nanocarrier of claim 1, wherein the nanocarrier comprises an average of from about 5 to about 14 cyclodextrin moieties.

46-64. (canceled)

65. A method of treating cancer in a patient, the method comprising administering a therapeutically effective amount of the nanocarrier of claim 10 to the patient, wherein the cancer is selected from the group consisting of bladder cancer, lung cancer, brain cancer, melanoma, gastrointestinal cancer, breast cancer, non-Hodgkin lymphoma, cervical cancer, ovarian cancer, colorectal cancer, pancreatic cancer, esophageal cancer, prostate cancer, kidney cancer, skin cancer, leukemia, thyroid cancer, liver cancer, and uterine cancer.

66-70. (canceled)

71. A method of imaging a tissue in a patient, comprising administering the nanocarrier of claim 7 to the patient, and imaging the patient with an imaging technique.

72. The method of claim 71, wherein the tissue comprises cancer cells.

73. The method of claim 72, wherein the tissue comprises kidney tissue, bladder tissue, or both.
